SEQUENCE LISTING (38 sequences)

SEQ ID NO: 1 - protein, 3 aa, Artificial
  Synthetic
SSG

SEQ ID NO: 2 - protein, 3 aa, Artificial
  Synthetic
SGG

SEQ ID NO: 3 - DNA, 12729 bp, Artificial
  Synthetic
gtccgggcagcccccggcgcagcgcggccgcagcagcctccgccccccgcacggtgtgagcgcccgacgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgactgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatgaccacggaggatagtatgagccctaaaaatccagactctttcgatacccaggaccaagccacagcaggtcctccatcccaacagccatgcccgcattagctcttagacccacagactggttttgcaacgtttacaccgactagccaggaagtacttccacctcgggcacattttgggaagttgcattcctttgtcttcaaactgtgaagcatttacagaaacgcatccagcaagaatattgtccctttgagcagaaatttatctttcaaagaggtatatttgaaaaaaaaaaaaagtatatgtgaggatttttattgattggggatcttggagtttttcattgtcgctattgatttttacttcaatgggctcttccaacaaggaagaagcttgctggtagcacttgctaccctgagttcatccaggcccaactgtgagcaaggagcacaagccacaagtcttccagaggatgcttgattccagtggttctgcttcaaggcttccactgcaaaacactaaagatccaagaaggccttcatggccccagcaggccggatcggtactgtatcaagtcatggcaggtacagtaggataagccactctgtcccttcctgggcaaagaagaaacggaggggatggaattcttccttagacttacttttgtaaaaatgtccccacggtacttactccccactgatggaccagtggtttccagtcatgagcgttagactgacttgtttgtcttccattccattgttttgaaactcagtatgctgcccctgtcttgctgtcatgaaatcagcaagagaggatgacacatcaaataataactcggattccagcccacattggattcatcagcatttggaccaatagcccacagctgagaatgtggaatacctaaggatagcaccgcttttgttctcgcaaaaacgtatctcctaatttgaggctcagatgaaatgcatcaggtcctttggggcatagatcagaagactacaaaaatgaagctgctctgaaatctcctttagccatcaccccaaccccccaaaattagtttgtgttacttatggaagatagttttctccttttacttcacttcaaaagctttttactcaaagagtatatgttccctccaggtcagctgcccccaaaccccctccttacgctttgtcacacaaaaagtgtctctgccttgagtcatctattcaagcacttacagctctggccacaacagggcattttacaggtgcgaatgacagtagcattatgagtagtgtggaattcaggtagtaaatatgaaactagggtttgaaattgataatgctttcacaacatttgcagatgttttagaaggaaaaaagttccttcctaaaataatttctctacaattggaagattggaagattcagctagttaggagcccaccttttttcctaatctgtgtgtgccctgtaacctgactggttaacagcagtcctttgtaaacagtgttttaaactctcctagtcaatatccaccccatccaatttatcaaggaagaaatggttcagaaaatattttcagcctacagttatgttcagtcacacacacatacaaaatgttccttttgcttttaaagtaatttttgactcccagatcagtcagagcccctacagcattgttaagaaagtatttgatttttgtctcaatgaaaataaaactatattcatttccactctattatgctctcaaatacccctaagcatctatactagcctggtatgggtatgaaagatacaaagataaataaaacatagtccctgattctaagaaattcacaatttagcaaaggaaatggactcatagatgctaaccttaaaacaacgtgacaaatgccagacaggacccatcagccaggcactgtgagagcacagagcagggaggttgggtcctgcctgaggagacctggaagggaggcctcacaggaggatgaccaggtctcagtcagcggggaggtggaaagtgcaggtgcatcaggggcaccctgaccgaggaaacagctgccagaggcctccactgctaaagtccacataaggctgaggtcagtcaccctaaacaacctgctccctctaagccaggggatgagcttggagcatcccacaagttccctaaaagttgcagcccccagggggattttgagctatcatctctgcacatgcttagtgagaagactacacaacatttctaagaatctgagattttatattgtcagttaaccactttcattattcattcacctcaggacatgcagaaatatttcagtcagaactgggaaacagaaggacctacattctgctgtcacttatgtgtcaagaagcagatgatcgatgaggcaggtcagttgtaagtgagtcacattgtagcattaaattctagtatttttgtagtttgaaacagtaacttaataaaagagcaaaagctaaaaaaaagtccgggcagcccccggcgcagcgcggccgcagcagcctccgccccccgcacggtgtgagcgcccgacgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgactgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatgaccacggaggatagtatgagccctaaaaatccagactctttcgatacccaggaccaagccacagcaggtcctccatcccaacagccatgcccgcattagctcttagacccacagactggttttgcaacgtttacaccgactagccaggaagtacttccacctcgggcacattttgggaagttgcattcctttgtcttcaaactgtgaagcatttacagaaacgcatccagcaagaatattgtccctttgagcagaaatttatctttcaaagaggtatatttgaaaaaaaaaaaaagtatatgtgaggatttttattgattggggatcttggagtttttcattgtcgctattgatttttacttcaatgggctcttccaacaaggaagaagcttgctggtagcacttgctaccctgagttcatccaggcccaactgtgagcaaggagcacaagccacaagtcttccagaggatgcttgattccagtggttctgcttcaaggcttccactgcaaaacactaaagatccaagaaggccttcatggccccagcaggccggatcggtactgtatcaagtcatggcaggtacagtaggataagccactctgtcccttcctgggcaaagaagaaacggaggggatggaattcttccttagacttacttttgtaaaaatgtccccacggtacttactccccactgatggaccagtggtttccagtcatgagcgttagactgacttgtttgtcttccattccattgttttgaaactcagtatgctgcccctgtcttgctgtcatgaaatcagcaagagaggatgacacatcaaataataactcggattccagcccacattggattcatcagcatttggaccaatagcccacagctgagaatgtggaatacctaaggatagcaccgcttttgttctcgcaaaaacgtatctcctaatttgaggctcagatgaaatgcatcaggtcctttggggcatagatcagaagactacaaaaatgaagctgctctgaaatctcctttagccatcaccccaaccccccaaaattagtttgtgttacttatggaagatagttttctccttttacttcacttcaaaagctttttactcaaagagtatatgttccctccaggtcagctgcccccaaaccccctccttacgctttgtcacacaaaaagtgtctctgccttgagtcatctattcaagcacttacagctctggccacaacagggcattttacaggtgcgaatgacagtagcattatgagtagtgtggaattcaggtagtaaatatgaaactagggtttgaaattgataatgctttcacaacatttgcagatgttttagaaggaaaaaagttccttcctaaaataatttctctacaattggaagattggaagattcagctagttaggagcccaccttttttcctaatctgtgtgtgccctgtaacctgactggttaacagcagtcctttgtaaacagtgttttaaactctcctagtcaatatccaccccatccaatttatcaaggaagaaatggttcagaaaatattttcagcctacagttatgttcagtcacacacacatacaaaatgttccttttgcttttaaagtaatttttgactcccagatcagtcagagcccctacagcattgttaagaaagtatttgatttttgtctcaatgaaaataaaactatattcatttccactctattatgctctcaaatacccctaagcatctatactagcctggtatgggtatgaaagatacaaagataaataaaacatagtccctgattctaagaaattcacaatttagcaaaggaaatggactcatagatgctaaccttaaaacaacgtgacaaatgccagacaggacccatcagccaggcactgtgagagcacagagcagggaggttgggtcctgcctgaggagacctggaagggaggcctcacaggaggatgaccaggtctcagtcagcggggaggtggaaagtgcaggtgcatcaggggcaccctgaccgaggaaacagctgccagaggcctccactgctaaagtccacataaggctgaggtcagtcaccctaaacaacctgctccctctaagccaggggatgagcttggagcatcccacaagttccctaaaagttgcagcccccagggggattttgagctatcatctctgcacatgcttagtgagaagactacacaacatttctaagaatctgagattttatattgtcagttaaccactttcattattcattcacctcaggacatgcagaaatatttcagtcagaactgggaaacagaaggacctacattctgctgtcacttatgtgtcaagaagcagatgatcgatgaggcaggtcagttgtaagtgagtcacattgtagcattaaattctagtatttttgtagtttgaaacagtaacttaataaaagagcaaaagctaaaaaaaaaaaaaaaaa

SEQ ID NO: 4 - protein, 1210 aa, Artificial [sequence omitted]

SEQ ID NO: 5 - DNA, 5700 bp, Artificial
  Synthetic
aaaaagagaaactgttgggagaggaatcgtatctccatatttcttctttcagccccaatccaagggttgtagctggaactttccatcagttcttcctttctttttcctctctaagcctttgccttgctctgtcacagtgaagtcagccagagcagggctgttaaactctgtgaaatttgtcataagggtgtcaggtatttcttactggcttccaaagaaacatagataaagaaatctttcctgtggcttcccttggcaggctgcattcagaaggtctctcagttgaagaaagagcttggaggacaacagcacaacaggagagtaaaagatgccccagggctgaggcctccgctcaggcagccgcatctggggtcaatcatactcaccttgcccgggccatgctccagcaaaatcaagctgttttcttttgaaagttcaaactcatcaagattatgctgctcactcttatcattctgttgccagtagtttcaaaatttagttttgttagtctctcagcaccgcagcactggagctgtcctgaaggtactctcgcaggaaatgggaattctacttgtgtgggtcctgcacccttcttaattttctcccatggaaatagtatctttaggattgacacagaaggaaccaattatgagcaattggtggtggatgctggtgtctcagtgatcatggattttcattataatgagaaaagaatctattgggtggatttagaaagacaacttttgcaaagagtttttctgaatgggtcaaggcaagagagagtatgtaatatagagaaaaatgtttctggaatggcaataaattggataaatgaagaagttatttggtcaaatcaacaggaaggaatcattacagtaacagatatgaaaggaaataattcccacattcttttaagtgctttaaaatatcctgcaaatgtagcagttgatccagtagaaaggtttatattttggtcttcagaggtggctggaagcctttatagagcagatctcgatggtgtgggagtgaaggctctgttggagacatcagagaaaataacagctgtgtcattggatgtgcttgataagcggctgttttggattcagtacaacagagaaggaagcaattctcttatttgctcctgtgattatgatggaggttctgtccacattagtaaacatccaacacagcataatttgtttgcaatgtccctttttggtgaccgtatcttctattcaacatggaaaatgaagacaatttggatagccaacaaacacactggaaaggacatggttagaattaacctccattcatcatttgtaccacttggtgaactgaaagtagtgcatccacttgcacaacccaaggcagaagatgacacttgggagcctgagcagaaactttgcaaattgaggaaaggaaactgcagcagcactgtgtgtgggcaagacctccagtcacacttgtgcatgtgtgcagagggatacgccctaagtcgagaccggaagtactgtgaagatgttaatgaatgtgctttttggaatcatggctgtactcttgggtgtaaaaacacccctggatcctattactgcacgtgccctgtaggatttgttctgcttcctgatgggaaacgatgtcatcaacttgtttcctgtccacgcaatgtgtctgaatgcagccatgactgtgttctgacatcagaaggtcccttatgtttctgtcctgaaggctcagtgcttgagagagatgggaaaacatgtagcggttgttcctcacccgataatggtggatgtagccagctctgcgttcctcttagcccagtatcctgggaatgtgattgctttcctgggtatgacctacaactggatgaaaaaagctgtgcagcttcaggaccacaaccatttttgctgtttgccaattctcaagatattcgacacatgcattttgatggaacagactatggaactctgctcagccagcagatgggaatggtttatgccctagatcatgaccctgtggaaaataagatatactttgcccatacagccctgaagtggatagagagagctaatatggatggttcccagcgagaaaggcttattgaggaaggagtagatgtgccagaaggtcttgctgtggactggattggccgtagattctattggacagacagagggaaatctctgattggaaggagtgatttaaatgggaaacgttccaaaataatcactaaggagaacatctctcaaccacgaggaattgctgttcatccaatggccaagagattattctggactgatacagggattaatccacgaattgaaagttcttccctccaaggccttggccgtctggttatagccagctctgatctaatctggcccagtggaataacgattgacttcttaactgacaagttgtactggtgcgatgccaagcagtctgtgattgaaatggccaatctggatggttcaaaacgccgaagacttacccagaatgatgtaggtcacccatttgctgtagcagtgtttgaggattatgtgtggttctcagattgggctatgccatcagtaatgagagtaaacaagaggactggcaaagatagagtacgtctccaaggcagcatgctgaagccctcatcactggttgtggttcatccattggcaaaaccaggagcagatccctgcttatatcaaaacggaggctgtgaacatatttgcaaaaagaggcttggaactgcttggtgttcgtgtcgtgaaggttttatgaaagcctcagatgggaaaacgtgtctggctctggatggtcatcagctgttggcaggtggtgaagttgatctaaagaaccaagtaacaccattggacatcttgtccaagactagagtgtcagaagataacattacagaatctcaacacatgctagtggctgaaatcatggtgtcagatcaagatgactgtgctcctgtgggatgcagcatgtatgctcggtgtatttcagagggagaggatgccacatgtcagtgtttgaaaggatttgctggggatggaaaactatgttctgatatagatgaatgtgagatgggtgtcccagtgtgcccccctgcctcctccaagtgcatcaacaccgaaggtggttatgtctgccggtgctcagaaggctaccaaggagatgggattcactgtcttgatattgatgagtgccaactgggggagcacagctgtggagagaatgccagctgcacaaatacagagggaggctatacctgcatgtgtgctggacgcctgtctgaaccaggactgatttgccctgactctactccaccccctcacctcagggaagatgaccaccactattccgtaagaaatagtgactctgaatgtcccctgtcccacgatgggtactgcctccatgatggtgtgtgcatgtatattgaagcattggacaagtatgcatgcaactgtgttgttggctacatcggggagcgatgtcagtaccgagacctgaagtggtgggaactgcgccacgctggccacgggcagcagcagaaggtcatcgtggtggctgtctgcgtggtggtgcttgtcatgctgctcctcctgagcctgtggggggcccactactacaggactcagaagctgctatcgaaaaacccaaagaatccttatgaggagtcgagcagagatgtgaggagtcgcaggcctgctgacactgaggatgggatgtcctcttgccctcaaccttggtttgtggttataaaagaacaccaagacctcaagaatgggggtcaaccagtggctggtgaggatggccaggcagcagatgggtcaatgcaaccaacttcatggaggcaggagccccagttatgtggaatgggcacagagcaaggctgctggattccagtatccagtgataagggctcctgtccccaggtaatggagcgaagctttcatatgccctcctatgggacacagacccttgaagggggtgtcgagaagccccattctctcctatcagctaacccattatggcaacaaagggccctggacccaccacaccaaatggagctgactcagtgaaaactggaattaaaaggaaagtcaagaagaatgaactatgtcgatgcacagtatcttttctttcaaaagtagagcaaaactataggttttggttccacaatctctacgactaatcacctactcaatgcctggagacagatacgtagttgtgcttttgtttgctcttttaagcagtctcactgcagtcttatttccaagtaagagtactgggagaatcactaggtaacttattagaaacccaaattgggacaacagtgctttgtaaattgtgttgtcttcagcagtcaatacaaatagatttttgtttttgttgttcctgcagccccagaagaaattaggggttaaagcagacagtcacactggtttggtcagttacaaagtaatttctttgatctggacagaacatttatatcagtttcatgaaatgattggaatattacaataccgttaagatacagtgtaggcatttaactcctcattggcgtggtccatgctgatgattttgcaaaatgagttgtgatgaatcaatgaaaaatgtaatttagaaactgatttcttcagaattagatggcttattttttaaaatatttgaatgaaaacattttatttttaaaatattacacaggaggcttcggagtttcttagtcattactgtccttttcccctacagaattttccctcttggtgtgattgcacagaatttgtatgtattttcagttacaagattgtaagtaaattgcctgatttgttttcattatagacaacgatgaatttcttctaattatttaaataaaatcaccaaaaacataaacattttattgtatgcctgattaagtagttaattatagtctaaggcagtactagagttgaaccaaaatgatttgtcaagcttgctgatgtttctgtttttcgttttttttttttttccggagagaggataggatctcactctgttatccaggctggagtgtgcaatggcacaatcatagctcagtgcagcctcaaactcctgggctcaagcaatcctcctgcctcagcctcccgagtaactaggaccacaggcacaggccaccatgcctggctaaggtttttatttttattttttgtagacatggggatcacacaatgttgcccaggctggtcttgaactcctggcctcaagcaaggtcgtgctggtaattttgcaaaatgaattgtgattgactttcagcctcccaacgtattagattataggcattagccatggtgcccagccttgtaacttttaaaaaaattttttaatctacaactctgtagattaaaatttcacatggtgttctaattaaatatttttcttgcagccaagatattgttactacagataacacaacctgatatggtaactttaaattttgggggctttgaatcattcagtttatgcattaactagtccctttgtttatctttcatttctcaaccccttgtactttggtgataccagacatcagaataaaaagaaattgaagtacctgttttcaaatggatactttataggaattttggtaaagatttggtgatgggaggatgacttgaggtttgtggatattagttaattattcagtatgatacctcacccagctaattt

SEQ ID NO: 6 - protein, 1207 aa, Artificial
  Synthetic
MLLTLIILLPVVSKFSFVSLSAPQHWSCPEGTLAGNGNSTCVGPAPFLIFSHGNSIFRIDTEGTNYEQLVVDAGVSVIMDFHYNEKRIYWVDLERQLLQRVFLNGSRQERVCNIEKNVSGMAINWINEEVIWSNQQEGIITVTDMKGNNSHILLSALKYPANVAVDPVERFIFWSSEVAGSLYRADLDGVGVKALLETSEKITAVSLDVLDKRLFWIQYNREGSNSLICSCDYDGGSVHISKHPTQHNLFAMSLFGDRIFYSTWKMKTIWIANKHTGKDMVRINLHSSFVPLGELKVVHPLAQPKAEDDTWEPEQKLCKLRKGNCSSTVCGQDLQSHLCMCAEGYALSRDRKYCEDVNECAFWNHGCTLGCKNTPGSYYCTCPVGFVLLPDGKRCHQLVSCPRNVSECSHDCVLTSEGPLCFCPEGSVLERDGKTCSGCSSPDNGGCSQLCVPLSPVSWECDCFPGYDLQLDEKSCAASGPQPFLLFANSQDIRHMHFDGTDYGTLLSQQMGMVYALDHDPVENKIYFAHTALKWIERANMDGSQRERLIEEGVDVPEGLAVDWIGRRFYWTDRGKSLIGRSDLNGKRSKIITKENISQPRGIAVHPMAKRLFWTDTGINPRIESSSLQGLGRLVIASSDLIWPSGITIDFLTDKLYWCDAKQSVIEMANLDGSKRRRLTQNDVGHPFAVAVFEDYVWFSDWAMPSVMRVNKRTGKDRVRLQGSMLKPSSLVVVHPLAKPGADPCLYQNGGCEHICKKRLGTAWCSCREGFMKASDGKTCLALDGHQLLAGGEVDLKNQVTPLDILSKTRVSEDNITESQHMLVAEIMVSDQDDCAPVGCSMYARCISEGEDATCQCLKGFAGDGKLCSDIDECEMGVPVCPPASSKCINTEGGYVCRCSEGYQGDGIHCLDIDECQLGEHSCGENASCTNTEGGYTCMCAGRLSEPGLICPDSTPPPHLREDDHHYSVRNSDSECPLSHDGYCLHDGVCMYIEALDKYACNCVVGYIGERCQYRDLKWWELRHAGHGQQQKVIVVAVCVVVLVMLLLLSLWGAHYYRTQKLLSKNPKNPYEESSRDVRSRRPADTEDGMSSCPQPWFVVIKEHQDLKNGGQPVAGEDGQAADGSMQPTSWRQEPQLCGMGTEQGCWIPVSSDKGSCPQVMERSFHMPSYGTQTLEGGVEKPHSLLSANPLWQQRALDPPHQMELTQ

SEQ ID NO: 7 - DNA, 2106 bp, Artificial
  Synthetic
gagcccttggaccaaactcgcctgcgccgagagccgtccgcgtagagcgctccgtctccggcgagatgtccgagcgcaaagaaggcagaggcaaagggaagggcaagaagaaggagcgaggctccggcaagaagccggagtccgcggcgggcagccagagcccagccttgcctccccaattgaaagagatgaaaagccaggaatcggctgcaggttccaaactagtccttcggtgtgaaaccagttctgaatactcctctctcagattcaagtggttcaagaatgggaatgaattgaatcgaaaaaacaaaccacaaaatatcaagatacaaaaaaagccagggaagtcagaacttcgcattaacaaagcatcactggctgattctggagagtatatgtgcaaagtgatcagcaaattaggaaatgacagtgcctctgccaatatcaccatcgtggaatcaaacgagatcatcactggtatgccagcctcaactgaaggagcatatgtgtcttcagagtctcccattagaatatcagtatccacagaaggagcaaatacttcttcatctacatctacatccaccactgggacaagccatcttgtaaaatgtgcggagaaggagaaaactttctgtgtgaatggaggggagtgcttcatggtgaaagacctttcaaacccctcgagatacttgtgcaagtgccaacctggattcactggagcaagatgtactgagaatgtgcccatgaaagtccaaaaccaagaaaaggcggaggagctgtaccagaagagagtgctgaccataaccggcatctgcatcgccctccttgtggtcggcatcatgtgtttggtggcctactgcaaaaccaagaaacagcggaaaaagctgcatgaccgtcttcggcagagccttcggtctgaacgaaacaatatgatgaacattgccaatgggcctcaccatcctaacccaccccccgagaatgtccagctggtgaatcaatacgtatctaaaaacgtcatctccagtgagcatattgttgagagagaagcagagacatccttttccaccagtcactatacttccacagcccatcactccactactgtcacccagactcctagccacagctggagcaacggacacactgaaagcatcctttccgaaagccactctgtaatcgtgatgtcatccgtagaaaacagtaggcacagcagcccaactgggggcccaagaggacgtcttaatggcacaggaggccctcgtgaatgtaacagcttcctcaggcatgccagagaaacccctgattcctaccgagactctcctcatagtgaaaggtatgtgtcagccatgaccaccccggctcgtatgtcacctgtagatttccacacgccaagctcccccaaatcgcccccttcggaaatgtctccacccgtgtccagcatgacggtgtccatgccttccatggcggtcagccccttcatggaagaagagagacctctacttctcgtgacaccaccaaggctgcgggagaagaagtttgaccatcaccctcagcagttcagctccttccaccacaaccccgcgcatgacagtaacagcctccctgctagccccttgaggatagtggaggatgaggagtatgaaacgacccaagagtacgagccagcccaagagcctgttaagaaactcgccaatagccggcgggccaaaagaaccaagcccaatggccacattgctaacagattggaagtggacagcaacacaagctcccagagcagtaactcagagagtgaaacagaagatgaaagagtaggtgaagatacgcctttcctgggcatacagaaccccctggcagccagtcttgaggcaacacctgccttccgcctggctgacagcaggactaacccagcaggccgcttctcgacacaggaagaaatccaggccaggctgtctagtgtaattgctaaccaagaccctattgctgtataaaacctaaataaacacatagattcacctgtaaaactttattttatataataaagtattccaccttaaattaaacaatttattttattttagcagttctgcaaatagaaaacaggaaaaa

SEQ ID NO: 8 - protein, 640 aa, Artificial [sequence omitted]

SEQ ID NO: 9 - DNA, 6661 bp, Artificial
  Synthetic
ggcttaactgatgcctgcctgcctctctttgatttgatggcctttattccttctaattggataaaataggaagtcactggcagtcctgtgtggctggggatactgattttactcagaccagcctgcagctctagagtgtgggtagagagcggggagtgggggttgggagagggggaggaaagagagagaggagagaggacgggcttggatgaagaagggaaagaaagagaaagagactgaagcagagaagagccgcagaggaagaaagtgaatgagcactcaagaaggacaaagaggagtagtcgggggtggggtggaggcagggcggggaagggagtgaccgcccctcctggctgcactcttgcctccggagccctctgatcctgtttgcagtgatgctccgagggcaggcacctgctgctctgtaatgattcagcccctttcagccgtcgtcgcgttaacacaacaggatgctgttgctattgtcactactgcctctcctgccgccgctgctgctgccgccgccgccaccgccgctggtcctccttctgcttttacttctcctgcatgacagttgttttcttcatctgagcagacaccagcttcagatgctcgaggtgagaaacatgcctttcagtttgggctactggtttacttaattaatcagccggcagctccgtcgatctattttcgtccctgtcctcttgacgagcccgggatggtttggagtagcatttaaaagaactagaaaagtggcccagaaacagcagcttaaagaattattacgatatactttgattttgtagttgctaggagcttttcttccccccttgcatctttctgaactcttcttgattttaataatggccttggacttggacgatttatcgatttccccctgtaagatgctgtatcatttggttgggggggcctctgcgtggtaatggaccgtgagagcggccaggccttcttctggaggtgagccgatggagatttattccccagacatgtctgaggtcgccgccgagaggtcctccagcccctccactcagctgagtgcagacccatctcttgatgggcttccggcagcagaagacatgccagagccccagactgaagatgggagaacccctggactcgtgggcctggccgtgccctgctgtgcgtgcctagaagctgagcgcctgagaggttgcctcaactcagagaaaatctgcattgtccccatcctggcttgcctggtcagcctctgcctctgcatcgccggcctcaagtgggtatttgtggacaagatctttgaatatgactctcctactcaccttgaccctggggggttaggccaggaccctattatttctctggacgcaactgctgcctcagctgtgtgggtgtcgtctgaggcatacacttcacctgtctctagggctcaatctgaaagtgaggttcaagttacagtgcaaggtgacaaggctgttgtctcctttgaaccatcagcggcaccgacaccgaagaatcgtatttttgccttttctttcttgccgtccactgcgccatccttcccttcacccacccggaaccctgaggtgagaacgcccaagtcagcaactcagccacaaacaacagaaactaatctccaaactgctcctaaactttctacatctacatccaccactgggacaagccatcttgtaaaatgtgcggagaaggagaaaactttctgtgtgaatggaggggagtgcttcatggtgaaagacctttcaaacccctcgagatacttgtgcaagtgcccaaatgagtttactggtgatcgctgccaaaactacgtaatggccagcttctacaagcatcttgggattgaatttatggaggcggaggagctgtaccagaagagagtgctgaccataaccggcatctgcatcgccctccttgtggtcggcatcatgtgtgtggtggcctactgcaaaaccaagaaacagcggaaaaagctgcatgaccgtcttcggcagagccttcggtctgaacgaaacaatatgatgaacattgccaatgggcctcaccatcctaacccaccccccgagaatgtccagctggtgaatcaatacgtatctaaaaacgtcatctccagtgagcatattgttgagagagaagcagagacatccttttccaccagtcactatacttccacagcccatcactccactactgtcacccagactcctagccacagctggagcaacggacacactgaaagcatcctttccgaaagccactctgtaatcgtgatgtcatccgtagaaaacagtaggcacagcagcccaactgggggcccaagaggacgtcttaatggcacaggaggccctcgtgaatgtaacagcttcctcaggcatgccagagaaacccctgattcctaccgagactctcctcatagtgaaaggtatgtgtcagccatgaccaccccggctcgtatgtcacctgtagatttccacacgccaagctcccccaaatcgcccccttcggaaatgtctccacccgtgtccagcatgacggtgtccatgccttccatggcggtcagccccttcatggaagaagagagacctctacttctcgtgacaccaccaaggctgcgggagaagaagtttgaccatcaccctcagcagttcagctccttccaccacaaccccgcgcatgacagtaacagcctccctgctagccccttgaggatagtggaggatgaggagtatgaaacgacccaagagtacgagccagcccaagagcctgttaagaaactcgccaatagccggcgggccaaaagaaccaagcccaatggccacattgctaacagattggaagtggacagcaacacaagctcccagagcagtaactcagagagtgaaacagaagatgaaagagtaggtgaagatacgcctttcctgggcatacagaaccccctggcagccagtcttgaggcaacacctgccttccgcctggctgacagcaggactaacccagcaggccgcttctcgacacaggaagaaatccaggccaggctgtctagtgtaattgctaaccaagaccctattgctgtataaaacctaaataaacacatagattcacctgtaaaactttattttatataataaagtattccaccttaaattaaacaatttattttattttagcagttctgcaaatagaaaacaggaaaaaaacttttataaattaaatatatgtatgtaaaaatgtgttatgtgccatatgtagcaattttttacagtatttcaaaacgagaaagatatcaatggtgcctttatgttatgttatgtcgagagcaagttttgtacagttacagtgattgcttttccacagtatttctgcaaaacctctcatagattcagtttttgctggcttcttgtgcattgcattatgatgttgactggatgtatgatttgcaagacttgcaactgtccctctgtttgcttgtagtagcacccgatcagtatgtcttgtaatggcacatccatccagatatgcctctcttgtgtatgaagttttctttgctttcagaatatgaaatgagttgtgtctactctgccagccaaaggtttgcctcattgggctctgagataatagtagatccaacagcatgctactattaaatacagcaagaaactgcattaagtaatgttaaatattaggaagaaagtaatactgtgatttaaaaaaaactatattattaatcagaagacagcttgctcttactaaaaggagctctcatttactttatttgattttatttttcttgacaaaaagcaacagttttagggatagcttagaaaatgggttctggcttgctatcagggtaaatctaacaccttacaagaggactgagtgtcactttctctctgggggaatgatccagcagcttatctagttgacaatcaaaacacggctgataaaggtgcaatcatttctgacatgtatttttcactgattttgaagctagtgattggttgtgtcttcttggctcaaaaagaagcatattacggcacaaaaagcccagcccagacagcacatgcagcattttgtctgaaatacttctagagtcaaacgtgcctgctgtacatagcgatgacttgtcatcatagggaagtatttccatcgtagagtgttcagaaggagtgactgtataggtggagagaagcttagtgactccgttgaaattttaaaatgtggatgaccacccctttctcccccttatttttcttttatctttccatgttgccttgatcaggtcataactatgcatgaacattttttatcaggaatggccgatgtgtatgtgatttgtaatcacaagtaatgattcatcaggaaatgtcaatcctgttggaaagattgcacctttacttgcagaagtgacccccacctgtgtcctgacctctccatttacaggctctctcacccatttcccccacctcctttaatttttgctttactgtcataaagtaggactaagattggtctaagcattgcatgttcttttgtgatggtaaatccaaaggaaggcctataagtattaacatttgaaataactgctaattcaggaaaatggaagaaaaaaaattatttgaaacacagaacccatttcatggcctgcctgatatctgtgaaatcagggctggagctttacttaggattcacatggcctcctaggaaccatgggacaaatgggaaacaggttatcgggggattcatgaagtcagtgagagtaattgcttcttttttgcgggtgaactgaatgtatttcttcaccaaatcttgatgttaacaattaaaaagaagaaatgacatgcaagtaggtcttagcagaaaaatgcaggctgggcatgagtcatgttgttaccctcccacatgctcctacaatccacagagatgcctgtctgcaggttcttgaagttattgttagtatttggtatctcaaatttttcgtcactgttcacatgccactttctctgtgcacagtggtatcctcatttgctttttaacctacactgaggagtctttgtcaggttgcactgattttccaattctgcagtaatgagtaagctcacggcatggggaagaagacagtcagtccaatgaagttctctaaattattttaacattgcctttgaaggccttgactcatccttagctatttcaatgaagaaattcctaccatgaatttaaaaccctaaaaattctgtttcaaattctttgggcattggggtactcagatatcccattgtggaagaattttaagaataaatagaagtttctgttgagaaccatgagcaacatgtttcttacaatgagaattgctatgcattttaaaattgcaaatatatatgaaaattgaagacaagaggaaattgtatttctaacttgattctgatcactcacagaggtggcatattattatagttgggacatcctttgcacccttcataaaaaaggccagctgactgctcagcatcacctgccaaggccactagatttgtgtttacaggggtatctctgtgatgcttgtcacatcactcttgaccacctctgttaataaattccgacagtgcagtggcgatcggagtgtgaacttatgttcccagcatatggaaagctatcttaggttttaaggtagtagaaattgcccaggagtttgacagcaactttgtttcccgggtctaaaatcgtatcccactgaggtgtatgcagtggagcataatacatgcaaatacatgcaaaactccttttgtttcacctaagattcactttctatcttactttcccttcctgcctagtgtgacttttgcccccaagagtgcctggacagcattctagtttctacaaaatggtcctctgtgtaggtgaatgtgtcccaaacctgctatcactttcttgtttcagtgtgactgtcttgttagaggtgaagtttatccagggtaacttgctcactaactattcctttttatggcctggggttaaagggcgcatggctcacactggtgaaaataaggaaggcctggtcttatcttgtattaataatactggctgcattccaccagccagagatttctatctgcgaagacctatgaaacactgaagagaaatgtaggcagaaggaaatggccacatatcacaagttctattatatattcttttgtaaatacatattgtatattacttggatgttttcttatatcatttactgtctttttgagttaatgtcagtttttactctctcaacttactatgtaacattgtaaataacataatgtcctttattatttatatttaagcatctaacatatagagttgttttcatataagtttaagataaatgtcaaaaatatatgttcttttgtttttctttgctttaaaattatgtatcttttccttttcttttttttaagaataatttattgttcaggagaaagaatgtatatgtaactgaaactatctgaagaatgcacattgaaggccgtgaggtactgataaactaaagaatttattattcaaaatactaagcaataagtaattgtgatttatttaaagttttgtccattttccatgaaagacatactgcaataaaaatgctactctgtggaaaaaaaaaaaaaaaaaa

SEQ ID NO: 10 - protein, 700 aa, Artificial
  Synthetic
MEIYSPDMSEVAAERSSSPSTQLSADPSLDGLPAAEDMPEPQTEDGRTPGLVGLAVPCCACLEAERLRGCLNSEKICIVPILACLVSLCLCIAGLKWVFVDKIFEYDSPTHLDPGGLGQDPIISLDATAASAVWVSSEAYTSPVSRAQSESEVQVTVQGDKAVVSFEPSAAPTPKNRIFAFSFLPSTAPSFPSPTRNPEVRTPKSATQPQTTETNLQTAPKLSTSTSTTGTSHLVKCAEKEKTFCVNGGECFMVKDLSNPSRYLCKCPNEFTGDRCQNYVMASFYKHLGIEFMEAEELYQKRVLTITGICIALLVVGIMCVVAYCKTKKQRKKLHDRLRQSLRSERNNMMNIANGPHHPNPPPENVQLVNQYVSKNVISSEHIVEREAETSFSTSHYTSTAHHSTTVTQTPSHSWSNGHTESILSESHSVIVMSSVENSRHSSPTGGPRGRLNGTGGPRECNSFLRHARETPDSYRDSPHSERYVSAMTTPARMSPVDFHTPSSPKSPPSEMSPPVSSMTVSMPSMAVSPFMEEERPLLLVTPPRLREKKFDHHPQQFSSFHHNPAHDSNSLPASPLRIVEDEEYETTQEYEPAQEPVKKLANSRRAKRTKPNGHIANRLEVDSNTSSQSSNSESETEDERVGEDTPFLGIQNPLAASLEATPAFRLADSRTNPAGRFSTQEEIQARLSSVIANQDPIAV

SEQ ID NO: 11 - protein, 55 aa, Artificial
  Synthetic
GTSHLVKCPLSHEAYCVNGGECFMVKDLSNPSRYLCKCPNEFTGDRCQNYVMASF

SEQ ID NO: 12 - protein, 53 aa, Artificial
  Synthetic
NTENDCPLSHEAYCLHDGVCRFLVQEDKPACVCVVGYVGERCQFRDLRWWDAR

SEQ ID NO: 13 - protein, 173 aa, Artificial
  Synthetic
MTPQNITDLCAEYHNTQIHTLNDKIFSYTESLAGKREMAIITFKNGATFQVEVPGSQHIDSQKKAIERMKDTLRIAYLTEAKVEKLCVWNNKTPHAIAAISMANSSGGSGGGSGTFYDIETLKVIDEEWQRTQCHPIETLVDIFQEYPDEIEYIFKPSCVPLMRCGGCCNDEG

SEQ ID NO: 14 - protein, 172 aa, Artificial
  Synthetic
TPQNITDLCAEYHNTQIHTLNDKIFSYTESLAGKREMAIITFKNGATFQVEVPGSQHIDSQKKAIERMKDTLRIAYLTEAKVEKLCVWNNKTPHAIAAISMANSSGGSGGGSGTFYDIETLKVIDEEWQRTQCHPIETLVDIFQEYPDEIEYIFKPSCVPLMRCGGCCNDEG

SEQ ID NO: 15 - DNA, 3660 bp, Artificial
  Synthetic
gcggaggcttggggcagccgggtagctcggaggtcgtggcgctgggggctagcaccagcgctctgtcgggaggcgcagcggttaggtggaccggtcagcggactcaccggccagggcgctcggtgctggaatttgatattcattgatccgggttttatccctcttcttttttcttaaacatttttttttaaaactgtattgtttctcgttttaatttatttttgcttgccattccccacttgaatcgggccgacggcttggggagattgctctacttccccaaatcactgtggattttggaaaccagcagaaagaggaaagaggtagcaagagctccagagagaagtcgaggaagagagagacggggtcagagagagcgcgcgggcgtgcgagcagcgaaagcgacaggggcaaagtgagtgacctgcttttgggggtgaccgccggagcgcggcgtgagccctcccccttgggatcccgcagctgaccagtcgcgctgacggacagacagacagacaccgcccccagccccagctaccacctcctccccggccggcggcggacagtggacgcggcggcgagccgcgggcaggggccggagcccgcgcccggaggcggggtggagggggtcggggctcgcggcgtcgcactgaaacttttcgtccaacttctgggctgttctcgcttcggaggagccgtggtccgcgcgggggaagccgagccgagcggagccgcgagaagtgctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagccgcgcgcgctccccaggccctggcccgggcctcgggccggggaggaagagtagctcgccgaggcgccgaggagagcgggccgccccacagcccgagccggagagggagcgcgagccgcgccggccccggtcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaaaaatcagttcgaggaaagggaaaggggcaaaaacgaaagcgcaagaaatcccggtataagtcctggagcgtgtacgttggtgcccgctgctgtctaatgccctggagcctccctggcccccatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtgagccgggcaggaggaaggagcctccctcagggtttcgggaaccagatctctcaccaggaaagactgatacagaacgatcgatacagaaaccacgctgccgccaccacaccatcaccatcgacagaacagtccttaatccagaaacctgaaatgaaggaagaggagactctgcgcagagcactttgggtccggagggcgagactccggcggaagcattcccgggcgggtgacccagcacggtccctcttggaattggattcgccattttatttttcttgctgctaaatcaccgagcccggaagattagagagttttatttctgggattcctgtagacacacccacccacatacatacatttatatatatatatattatatatatataaaaataaatatctctattttatatatataaaatatatatattctttttttaaattaacagtgctaatgttattggtgtcttcactggatgtatttgactgctgtggacttgagttgggaggggaatgttcccactcagatcctgacagggaagaggaggagatgagagactctggcatgatcttttttttgtcccacttggtggggccagggtcctctcccctgcccaggaatgtgcaaggccagggcatgggggcaaatatgacccagttttgggaacaccgacaaacccagccctggcgctgagcctctctaccccaggtcagacggacagaaagacagatcacaggtacagggatgaggacaccggctctgaccaggagtttggggagcttcaggacattgctgtgctttggggattccctccacatgctgcacgcgcatctcgcccccaggggcactgcctggaagattcaggagcctgggcggccttcgcttactctcacctgcttctgagttgcccaggagaccactggcagatgtcccggcgaagagaagagacacattgttggaagaagcagcccatgacagctccccttcctgggactcgccctcatcctcttcctgctccccttcctggggtgcagcctaaaaggacctatgtcctcacaccattgaaaccactagttctgtccccccaggagacctggttgtgtgtgtgtgagtggttgaccttcctccatcccctggtccttcccttcccttcccgaggcacagagagacagggcaggatccacgtgcccattgtggaggcagagaaaagagaaagtgttttatatacggtacttatttaatatccctttttaattagaaattaaaacagttaatttaattaaagagtagggttttttttcagtattcttggttaatatttaatttcaactatttatgagatgtatcttttgctctctcttgctctcttatttgtaccggtttttgtatataaaattcatgtttccaatctctctctccctgatcggtgacagtcactagcttatcttgaacagatatttaattttgctaacactcagctctgccctccccgatcccctggctccccagcacacattcctttgaaataaggtttcaatatacatctacatactatatatatatttggcaacttgtatttgtgtgtatatatatatatatatgtttatgtatatatgtgattctgataaaatagacattgctattctgttttttatatgtaaaaacaaaacaagaaaaaatagagaattctacatactaaatctctctccttttttaattttaatatttgttatcatttatttattggtgctactgtttatccgtaataattgtggggaaaagatattaacatcacgtctttgtctctagtgcagtttttcgagatattccgtagtacatatttatttttaaacaacgacaaagaaatacagatatatcttaaaaaaaaaaaagcattttgtattaaagaatttaattctgatctcaaa

SEQ ID NO: 16 - protein, 412 aa, Artificial
  Synthetic
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 17 - DNA, 2069 bp, Artificial
  Synthetic
aagacacatgcttctgcaagcttccatgaaggttgtgcaaaaaagtttcaatccagagttgggttccagctttctgtagctgtaagcattggtggccacaccacctccttacaaagcaactagaacctgcggcatacattggagagatttttttaattttctggacatgaagtaaatttagagtgctttctaatttcaggtagaagacatgtccaccttctgattatttttggagaacattttgatttttttcatctctctctccccacccctaagattgtgcaaaaaaagcgtaccttgcctaattgaaataatttcattggattttgatcagaactgattatttggttttctgtgtgaagttttgaggtttcaaactttccttctggagaatgccttttgaaacaattttctctagctgcctgatgtcaactgcttagtaatcagtggatattgaaatattcaaaatgtacagagagtgggtagtggtgaatgttttcatgatgttgtacgtccagctggtgcagggctccagtaatgaacatggaccagtgaagcgatcatctcagtccacattggaacgatctgaacagcagatcagggctgcttctagtttggaggaactacttcgaattactcactctgaggactggaagctgtggagatgcaggctgaggctcaaaagttttaccagtatggactctcgctcagcatcccatcggtccactaggtttgcggcaactttctatgacattgaaacactaaaagttatagatgaagaatggcaaagaactcagtgcagccctagagaaacgtgcgtggaggtggccagtgagctggggaagagtaccaacacattcttcaagcccccttgtgtgaacgtgttccgatgtggtggctgttgcaatgaagagagccttatctgtatgaacaccagcacctcgtacatttccaaacagctctttgagatatcagtgcctttgacatcagtacctgaattagtgcctgttaaagttgccaatcatacaggttgtaagtgcttgccaacagccccccgccatccatactcaattatcagaagatccatccagatccctgaagaagatcgctgttcccattccaagaaactctgtcctattgacatgctatgggatagcaacaaatgtaaatgtgttttgcaggaggaaaatccacttgctggaacagaagaccactctcatctccaggaaccagctctctgtgggccacacatgatgtttgacgaagatcgttgcgagtgtgtctgtaaaacaccatgtcccaaagatctaatccagcaccccaaaaactgcagttgctttgagtgcaaagaaagtctggagacctgctgccagaagcacaagctatttcacccagacacctgcagctgtgaggacagatgcccctttcataccagaccatgtgcaagtggcaaaacagcatgtgcaaagcattgccgctttccaaaggagaaaagggctgcccaggggccccacagccgaaagaatccttgattcagcgttccaagttccccatccctgtcatttttaacagcatgctgctttgccaagttgctgtcactgtttttttcccaggtgttaaaaaaaaaatccattttacacagcaccacagtgaatccagaccaaccttccattcacaccagctaaggagtccctggttcattgatggatgtcttctagctgcagatgcctctgcgcaccaaggaatggagaggaggggacccatgtaatccttttgtttagttttgtttttgttttttggtgaatgagaaaggtgtgctggtcatggaatggcaggtgtcatatgactgattactcagagcagatgaggaaaactgtagtctctgagtcctttgctaatcgcaactcttgtgaattattctgattcttttttatgcagaatttgattcgtatgatcagtactgactttctgattactgtccagcttatagtcttccagtttaatgaactaccatctgatgtttcatatttaagtgtatttaaagaaaataaacaccattattcaagcca

SEQ ID NO: 18 - protein, 354 aa, Artificial
  Synthetic
MYREWVVVNVFMMLYVQLVQGSSNEHGPVKRSSQSTLERSEQQIRAASSLEELLRITHSEDWKLWRCRLRLKSFTSMDSRSASHRSTRFAATFYDIETLKVIDEEWQRTQCSPRETCVEVASELGKSTNTFFKPPCVNVFRCGGCCNEESLICMNTSTSYISKQLFEISVPLTSVPELVPVKVANHTGCKCLPTAPRHPYSIIRRSIQIPEEDRCSHSKKLCPIDMLWDSNKCKCVLQEENPLAGTEDHSHLQEPALCGPHMMFDEDRCECVCKTPCPKDLIQHPKNCSCFECKESLETCCQKHKLFHPDTCSCEDRCPFHTRPCASGKTACAKHCRFPKEKRAAQGPHSRKNP

SEQ ID NO: 19 - protein, 4 aa, Artificial
  Synthetic
GSSG

SEQ ID NO: 20 - protein, 5 aa, Artificial
  Synthetic
SSGGG

SEQ ID NO: 21 - protein, 5 aa, Artificial
  Synthetic
GGSGG

SEQ ID NO: 22 - protein, 5 aa, Artificial
  Synthetic
GGGGS

SEQ ID NO: 23 - protein, 8 aa, Artificial
  Synthetic
SSGGGSGG

SEQ ID NO: 24 - protein, 10 aa, Artificial
  Synthetic
SSGGGGSGGG

SEQ ID NO: 25 - protein, 7 aa, Artificial
  Synthetic
TSGGGSG

SEQ ID NO: 26 - protein, 9 aa, Artificial
  Synthetic
TSGGGGSGG

SEQ ID NO: 27 - protein, 10 aa, Artificial
  Synthetic
SSGGSGGGSG

SEQ ID NO: 28 - protein, 11 aa, Artificial
  Synthetic
SSGGGSGGSSG

SEQ ID NO: 29 - protein, 12 aa, Artificial
  Synthetic
GGSGGTSGGGSG

SEQ ID NO: 30 - protein, 12 aa, Artificial
  Synthetic
SGGTSGGGGSGG

SEQ ID NO: 31 - protein, 13 aa, Artificial
  Synthetic
SSGGGGSGGGSSG

SEQ ID NO: 32 - protein, 13 aa, Artificial
  Synthetic
SSGGGSGGSSGGG

SEQ ID NO: 33 - protein, 15 aa, Artificial
  Synthetic
SSGGGGSGGGSSGGG

SEQ ID NO: 34 - protein, 19 aa, Artificial
  Synthetic
TFYDIETLKVIDEEWQRTQ

SEQ ID NO: 35 - protein, 40 aa, Artificial
  Synthetic
CHPIETLVDIFQEYPDEIEYIFKPSCVPLMRCGGCCNDEG

SEQ ID NO: 36 - protein, 173 aa, Artificial; identical to SEQ ID NO: 13
  Synthetic

SEQ ID NO: 37 - protein, 172 aa, Artificial; identical to SEQ ID NO: 14
  Synthetic

SEQ ID NO: 38 - protein, 14 aa, Artificial
  Synthetic
GGSGGTSGGGGSGG

(19) United States
(12) Patent Application Publication
(10) Pub. No.: US 2022/0305105 A1
(43) Pub. Date: Sep. 29, 2022

(54) STABILIZED CHIMERIC SYNTHETIC PROTEINS AND THERAPEUTIC USES THEREOF

(71) Applicant: In3Bio Ltd., Hamilton (BM)

(72) Inventors: Erik D'Hondt, Bazel (BE); Keith Alan Charlton, Aberdeen (GB)

(73) Assignee: In3Bio Ltd., Hamilton (BM)

(21) Appl. No.: 17/618,065

(22) PCT Filed: Jun. 24, 2020
(86) PCT No.: PCT/IB2020/000538

§ 371 Date: Dec. 10, 2021

Related U.S. Application Data
(60) Provisional application No. 62/866,190, filed on Jun. 25, 2019.

(51) Int. Cl. A61K 39/00

(52) U.S. Cl. CPC A61K 39/001135; A61K 2039/6037; A61K 2039/627; A61K 2039/70

(57) ABSTRACT

The disclosure relates to compositions and methods for treating disease. More particularly, the disclosure relates to stabilized chimeric synthetic proteins and their use for treating cancer.

### FIELD OF THE DISCLOSURE

The disclosure relates to compositions and methods for treating disease. More particularly, the disclosure relates to stabilized chimeric synthetic proteins and their use for treating cancer.

### BACKGROUND OF THE DISCLOSURE

According to the World Health Organization, neoplasia (e.g., cancer) is one of the leading causes of death worldwide and was responsible for 8.8 million deaths in 2015. The frequency of cancer in the global human population is significant: accounting for nearly 1 in 6 deaths. In 2015, the most common cancer deaths occurred from the following types of cancer: lung cancer (about 1.7 million deaths), liver cancer (about 800,000 deaths), colorectal cancer (about 800,000 deaths), stomach cancer (about 800,000 deaths), and breast cancer (about 600,000 deaths).

Cancer is typically treated by any of a variety of methods such as, for example, surgery, chemotherapy, radiation therapy, cancer immunotherapy, and the like. Unfortunately, many of these methods have toxic/undesirable side effects. For example, standard cancer chemotherapies were developed based on their ability to kill rapidly dividing cells, and many have toxic properties that cause undesirable side effects such as, for example, immunosuppression, nausea, hair loss, and the like. A central goal of cancer research over the past two decades has been to identify new therapies with greater efficacy and fewer side effects.

One such therapy is encompassed by cancer immunology, which is the study of interactions between an immune system and cancer cells such as tumors or malignancies. The initiation of an immune response, such as recognition of cancer-specific antigens that are expressed by human tumors and not expressed in normal tissues, is of particular interest. Generally, methods to control the division and proliferation of the malignant cells have focused on isolating these antigens and presenting them so that they are recognized by the immune system as non-self antigens to induce a specific immune response (e.g., cancer vaccines). Such cancer vaccines may typically be created as either chemical conjugates or recombinant proteins. Disadvantageously, such cancer vaccines exhibit a number of significant limitations, which arise primarily from the method of manufacture and the potential lack of uniformity, activity, and homology of the protein product. For example, cancer vaccines generated by chemical conjugation (e.g., via glutaraldehyde) generally comprise a mixture of a recombinant carrier protein and polypeptides of human origin. Unfortunately, the use of glutaraldehyde as a cross-linking reagent has the undesirable tendency to form covalent cross-linking bonds between varieties of chemical groups, and generally leads to a highly heterogeneous product. Thus, the resulting vaccines may comprise not only carrier protein molecules with varying numbers of the target human polypeptide attached (e.g., 0, 1, 2, 3, etc.), but the human polypeptides can each be attached to the carrier via different atoms and therefore be present in different positions and in different orientations. Furthermore, both the target polypeptide and carrier protein molecules may be conjugated to themselves, resulting in various homo-multimers that may have no clinical efficacy and may not contribute to an anti-cancer patient immune response. Additionally, cancer vaccines generated by recombinant protein technology have the disadvantage that the target human polypeptides included within the recombinant protein may not be able to fold properly, thereby preventing a useful immune response. Accordingly, there is an urgent need for new cancer vaccines that overcome these significant existing limitations in the field of cancer immunotherapy.

### SUMMARY OF THE DISCLOSURE

The present disclosure is directed towards chimeric synthetic proteins/molecules and their respective methods of manufacturing; the characterization of the chimeric synthetic proteins/molecules and therapeutic methods of using the chimeric synthetic proteins/molecules to treat chronic diseases, such as, for example, lung, breast, bladder, prostate, ovarian, vulva, colonic, colorectal, intestinal, pulmonary, brain, esophageal, other cancers, and other diseases.

The present disclosure provides chimeric synthetic proteins that may be used as therapeutic modalities to treat diseases such as, for example, cancer. In an illustrative embodiment, the present disclosure provides a chimeric synthetic protein/molecule including one or more protein domains from a synthetic growth factor (e.g., VEGF), one or more linker regions, and one or more immunogenic domains. In one aspect, the present disclosure provides a chimeric synthetic protein that includes a chimeric polypeptide sequence; at least one linker, and a polypeptide sequence. Advantageously, the chimeric synthetic proteins/molecules described herein have the ability to function as stabilizing scaffolds that better enable human proteins (e.g., growth factors such as VEGF, EGF, TGF, and the like) that are incorporated into the proteins/molecules to adopt native configurations when expressed (e.g., fold properly). Additionally, the chimeric synthetic proteins/molecules described herein have the ability to generate stabilized chimeric synthetic proteins/molecules that have long storage shelf lives.

In some embodiments, the polypeptide sequence includes an immunogenic polypeptide sequence.

In some embodiments, the polypeptide sequence includes a cholera toxin B (CT-B) protein.

In some embodiments, the at least one linker includes a first linker that separates the chimeric polypeptide sequence from the polypeptide sequence.

In some embodiments, the first linker is selected from the group consisting of SSG, GSSG, SSGGG, SGG, GGSGG, GGGGS, SSGGGSGG, SSGGGGSGGG, TSGGGSG, TSGGGGSGG, SSGGSGGGSG, SSGGGSGGSSG, GGSGGTSGGGSG, SGGTSGGGGSGG, GGSGGTSGGGGSGG, SSGGGGSGGGSSG, SSGGGSGGSSGGG, and SSGGGGSGGGSSGGG.

In some embodiments, the first linker is SSGGSGGGSG.

In some embodiments, the chimeric polypeptide sequence includes a vascular endothelial growth factor (VEGF) sequence.

In some embodiments, the chimeric polypeptide sequence includes a VEGF sequence selected from the group consisting of VEGF-A, VEGF-B, VEGF-C, VEGF-D, and combinations thereof.

In some embodiments, the chimeric polypeptide sequence includes a first VEGF domain and a second VEGF domain.

In some embodiments, the first VEGF domain includes VEGF-D, or a portion thereof, and the second VEGF domain includes VEGF-A, or a portion thereof.

In some embodiments, the first VEGF domain is TFYDIETLKVIDEEWQRTQ and the second VEGF domain is CHPIETLVDIFQEYPDEIEYIFKPSCVPLMRCGGCCNDEG.

In some embodiments, the chimeric polypeptide sequence binds to a vascular endothelial growth factor receptor (VEGFR) selected from the group consisting of VEGFR-1, VEGFR-2, VEGFR-3, and combinations thereof.

In some embodiments, the chimeric polypeptide sequence binds to VEGFR-1, VEGFR-2, and VEGFR-3.

In some embodiments, the chimeric synthetic protein initially has an amino acid sequence of

MTPQNITDLCAEYHNTQIHTLNDKIFSYTESLAGKREMAIITFKNGATFQV
EVPGSQHIDSQKKAIERMKDTLRIAYLTEAKVEKLCVWNNKTPHAIAAISM
ANSSGGSGGGSGTFYDIETLKVIDEEWQRTQCHPIETLVDIFQEYPDEIEY
IFKPSCVPLMRCGGCCNDEG.

In some embodiments, the initial chimeric synthetic protein is processed to have an amino acid sequence of

TPQNITDLCAEYHNTQIHTLNDKIFSYTESLAGKREMAIITFKNGATFQVE
VPGSQHIDSQKKAIERMKDTLRIAYLTEAKVEKLCVWNNKTPHAIAAISMA
NSSGGSGGGSGTFYDIETLKVIDEEWQRTQCHPIETLVDIFQEYPDEIEYI
FKPSCVPLMRCGGCCNDEG.

In another aspect, the present disclosure provides an immunogenic composition that includes a chimeric polypeptide sequence; at least one linker, and a polypeptide sequence.

In some embodiments, the polypeptide sequence includes an immunogenic polypeptide sequence.

In some embodiments, the polypeptide sequence includes a cholera toxin B (CT-B) protein.

In some embodiments, the at least one linker includes a first linker that separates the chimeric polypeptide sequence from the polypeptide sequence.

In some embodiments, the first linker is selected from the group consisting of SSG, GSSG, SSGGG, SGG, GGSGG, GGGGS, SSGGGSGG, SSGGGGSGGG, TSGGGSG, TSGGGGSGG, SSGGSGGGSG, SSGGGSGGSSG, GGSGGTSGGGSG, SGGTSGGGGSGG, GGSGGTSGGGGSGG, SSGGGGSGGGSSG, SSGGGSGGSSGGG, and SSGGGGSGGGSSGGG.

In some embodiments, the first linker is SSGGSGGGSG.

In some embodiments, the chimeric polypeptide sequence includes a vascular endothelial growth factor (VEGF) sequence.

In some embodiments, the chimeric polypeptide sequence includes a VEGF sequence selected from the group consisting of VEGF-A, VEGF-B, VEGF-C, VEGF-D, and combinations thereof.

In some embodiments, the chimeric polypeptide sequence includes a first VEGF domain and a second VEGF domain.

In some embodiments, the first VEGF domain includes VEGF-D, or a portion thereof, and the second VEGF domain includes VEGF-A, or a portion thereof.

In some embodiments, the first VEGF domain is TFYDIETLKVIDEEWQRTQ and the second VEGF domain is CHPIETLVDIFQEYPDEIEYIFKPSCVPLMRCGGCCNDEG.

In some embodiments, the chimeric polypeptide sequence binds to a vascular endothelial growth factor receptor (VEGFR) selected from the group consisting of VEGFR-1, VEGFR-2, VEGFR-3, and combinations thereof.

In some embodiments, the chimeric polypeptide sequence binds to VEGFR-1, VEGFR-2, and VEGFR-3.

In some embodiments, the synthetic protein chimeric synthetic protein initially has an amino acid sequence of

MTPQNITDLCAEYHNTQIHTLNDKIFSYTESLAGKREMAIITFKNGATFQV
EVPGSQHIDSQKKAIERMKDTLRIAYLTEAKVEKLCVWNNKTPHAIAAISM
ANSSGGSGGGSGTFYDIETLKVIDEEWQRTQCHPIETLVDIFQEYPDEIEY
IFKPSCVPLMRCGGCCNDEG.

In some embodiments, the initial chimeric synthetic protein is processed to have an amino acid sequence of

TPQNITDLCAEYHNTQIHTLNDKIFSYTESLAGKREMAIITFKNGATFQVE
VPGSQHIDSQKKAIERMKDTLRIAYLTEAKVEKLCVWNNKTPHAIAAISMA
NSSGGSGGGSGTFYDIETLKVIDEEWQRTQCHPIETLVDIFQEYPDEIEYI
FKPSCVPLMRCGGCCNDEG.

In some embodiments, the immunogenic composition further comprises an adjuvant.

In another aspect, the present disclosure provides a method of treating a patient in need thereof, that includes the step of administering to the patient the above-described immunogenic composition in a same day or at alternate days or times during a vaccination period.

In some embodiments, the patient has a cancer.

### Definitions

By “Epidermal Growth Factor Receptor (EGFR) nucleic acid molecule” is meant a polynucleotide encoding an EGFR polypeptide. An exemplary EGFR nucleic acid molecule is provided at NCBI Accession No. NM_005228.4, and reproduced below (SEQ ID NO:3):

>NM_005228.4
gtccgggcagcccccggagcagcgcggccgcagcagcctccgccccccgcacggtgtgag
cgcccgacgcggccgaggcggccggagtcccgagctagccccggcggccgccgccgccca
gaccggacgacaggccccctcgtcggcgtccgcccgagtccccgcctcgccgccaacgcc
acaaccaccgcgcacggccccctgactccgtccagtattgatcgggagagccggagcgag
ctcttcggggagcagcgatgcgaccctccgggacggccggggcagcgctcctggcgctgc
tggctgcgctctgcccggcgagtcgggctctggaggaaaagaaagtttgccaaggcacga
gtaacaagctcacgcagttgggcacttttgaagatcattttctcagcctccagaggatgt
tcaataactgtgaggtggtccttgggaatttggaaattacctatgtgcagaggaattatg
atctttccttcttaaagaccatccaggaggtggctggttatgtcctcattgccctcaaca
cagtggagcgaattcctttggaaaacctgcagatcatcagaggaaatatgtactacgaaa
attcctatgccttagcagtcttatctaactatgatgcaaataaaaccggactgaaggagc
tgcccatgagaaatttacaggaaatcctgcatggcgccgtgcggttcagcaacaaccctg
ccctgtgcaacgtggagagcatccagtggcgggacatagtcagcagtgactttctcagca
acatgtcgatggacttccagaaccacctgggcagctgccaaaagtgtgatccaagctgtc
ccaatgggagctgctggggtgcaggagaggagaactgccagaaactgaccaaaatcatct
gtgcccagcagtgctccgggcgctgccgtggcaagtcccccagtgactgctgccacaacc
agtgtgctgcaggctgcacaggcccccgggagagcgactgcctggtctgccgcaaattcc
gagacgaagccacgtgcaaggacacctggcccccactcatgctctacaaccccaccacgt
accagatggatgtgaacccggagggcaaatacagctttggtgccacctgcgtgaagaagt
gtgggcgtaattatgtggtgacagatcacggctcgtgcgtgcgagcctgtggggccgaca
gctatgagatggaggaagacggcgtccgcaagtgtaagaagtgcgaagggccttgccgca
aagtgtgtaacggaataggtattggtgaatttaaagactcactctccataaatgctacga
atattaaacacttcaaaaactgcacctccatcagtggcgatctccacatcctgccggtgg
catttaggggtgactccttcacacatactcctcctctggatccacaggaactggatattc
tgaaaaccgtaaaggaaatcacagggtttttgctgattcaggcttggcctgaaaacagga
cggacctccatgcctttgagaacctagaaatcataagcggcaggaccaagcaacatggtc
agataagtgatggagatgtgataatttcaggaaacaaaaatttgtgctatgcaaatacaa
taaactggaaaaaactgtttgggacctccggtcagaaaaccaaaattataagcaacagag
gtgaaaacagctgcaaGgccacaggccaggtctgccatgccttgtgctcccccgagggct
gctggggcccggagcccagggactgcgtctcttgccggaatgtcagccgaggcagggaat
gcgtggacaagtgcaaccttctggagggtgagccaagggagtttgtggagaactctgagt
qcatacagtgccacccagagtgcctgcctcaggccatgaacatcacctgcacaggacggg
gaccagacaactgtatccagtgtgcccactacattgacggcccccactgcgtcaagacct
gcccggcaggagtcatgggagaaaacaacaccctggtctggaagtacgcagacgccggcc
atgtgtgccacctgtgccatccaaactgcaactacggatgcactgggccaggtcttgaag
gctgtccaacgaatgggcctaagatcccgtccatoqccactgggatggtgggggccctcc
tcttgctgctggtggtggccctggggatcggcctcttcatgcgaaggcgccacatcgttc
ggaagcgcacgctgcggaggctgctgcaggagacggagcttgtggagcctcttacaccca
gtggagaagctcccaaccaagctctcttgaggatcttgaaggaaactgaattcaaaaaga
tcaaagtgctgggctccggtgcgttcggcacggtgtataagggactctggatcccagaag
gtgagaaagttaaaattcccgtcgctatcaaggaattaagagaagcaacatctccgaaag
ccaacaaggaaatcctcgatgaagcctacgtgatggccagcgtggacaacccccacgtgt
gccgcctgctgggcatctgcctcacctccaccgtgcagctcatcacgcagctcatgccct
tcggctgcctcctggactgtgtccgggaacacaaagacaatattggctcccagtacctgc
tcaactggtgtgtgcagatcgcaaagggcatgaactacttggaggaccgtcgcttggtgc
accgcgacctggcagccaggaacgtactggtgaaaacaccgcagcatgtcaagatcacag
attttgggctggccaaactgctgggtgcggaagagaaagaataccatgcagaaggaggca
aagtgcctatcaagtggatggcattggaatcaattttacacagaatctatacccaccaga
gtgatgtctggagctacggggtgactgtttgggagttgatgacctttggatccaagccat
atgacggaatccctgccagcgagatctcctccatcctggagaaaggagaacgcctccctc
agccacccatatgtaccatcgatgtctacatgatcatggtcaagtgctggatgatagacg
cagatagtcgcccaaagttccgtgagttgatcatcgaattctccaaaatggcccgagacc
cccagcgctaccttgtcattcagggggatgaaagaatgcatttgccaagtcctacagact
ccaacttctaccgtgccctgatggatgaagaagacatggacgacgtggtggatgccgacg
agtacctcatcccacagcagggcttcttcagcagcccctccacgtcacggactcccctcc
tgagctctctgagtgcaaccagcaacaattccacagtggcttgcattgatagaaatgggc
tgcaaagctgtcccatcaaggaagacagcttcttgcagcgatacagctcagaccccacag
gcgccttgactgaggacagcatagacgacaccttcctcccagtgcctgaatacataaacc
agtccgttcccaaaaggcccgctggctctgtgcagaatcctgtctatcacaatcagcctc
tgaaccccgcgcccagcagagacccacactaccaggacccccacagcactgcagtgggca
accccgagtatctcaacactgtccagcccacctgtgtcaacagcacattcgacagccctg
cccactgggcccagaaaggcagccaccaaattagcctggacaaccctgactaccagcagg
acttctttcccaaggaagccaagccaaatggcatctttaagggctccacagctgaaaatg
cagaatacctaagggtcgcgccacaaagcagtgaatttattggagcatgaccacggagga
tagtatgagccctaaaaatccagactctttcgatacccaggaccaagccacagcaggtcc
tccatcccaacagccatgcccgcattagctcttagacccacagactggttttgcaacgtt
tacaccgactagccaggaagtacttccacctcgggcacattttgggaagttgcattcctt
tgtcttcaaactgtgaagcatttacagaaacgcatccagcaagaatattgtccctttgag
cagaaatttatctttcaaagaggtatatttgaaaaaaaaaaaaagtatatgtgaggattt
ttattgattggggatcttggagtttttcattgtcgctattgatttttacttcaatgggct
cttccaacaaggaagaagcttgctggtagcacttgctaccctgagttcatccagccccaa
ctgtgagcaaggagcacaagccacaagtcttccagaggatgcttgattccagtggttctg
cttcaaggcttccactgcaaaacactaaagatccaagaaggccttcatggccccagcagg
ccggatcggtactgtatcaagtcatggcaggtacagtaggataagccactctgtcccttc
ctgggcaaagaagaaacggaggggatggaattcttccttagacttacttttgtaaaaatg
tccccacggtacttactccccactgatggaccagtggtttccagtcatgagcgttagact
gacttgtttgtcttccattccattgttttgaaactcagtatgctgcccctgtcttgctgt
catgaaatcagcaagagaggatgacacatcaaataataactcggattccagcccacattg
gattcatcagcatttggaccaatagcccacagctgagaatgtggaatacctaaggatagc
accgcttttgttctcgcaaaaacgtatctcctaatttgaggctcagatgaaatgcatcag
gtcctttggggcatagatcagaagactacaaaaatgaagctgctctgaaatctcctttag
ccatcaccccaaccccccaaaattagtttgtgttacttatggaagatagttttctccttt
tacttcacttcaaaagctttttactcaaagagtatatgttccctccaggtcagctgcccc
caaaccccctccttacgctttgtcacacaaaaagtgtctctgccttgagtcatctattca
agcacttacagctctggccacaacagggcattttacaggtgcgaatgacagtagcattat
gagtagtgtggaattcaggtagtaaatatgaaactagggtttgaaattgataatgctttc
acaacatttgcagatgttttagaaggaaaaaagttccttcctaaaataatttctctacaa
ttggaagattggaagattcagctagttaggagcccaccttttttcctaatctgtgtgtgc
cctgtaacctgactggttaacagcagtcctttgtaaacagtgttttaaactctcctagtc
aatatccaccccatccaatttatcaaggaagaaatggttcagaaaatattttcagcctac
agttatgttcagtcacacacacatacaaaatgttccttttgcttttaaagtaatttttga
ctcccagatcagtcagagcccctacagcattgttaagaaagtatttgatttttgtctcaa
tgaaaataaaactatattcatttccactctattatgctctcaaatacccctaagcatcta
tactagcctggtatgggtatgaaagatacaaagataaataaaacatagtccctgattcta
agaaattcacaatttagcaaaggaaatggactcatagatgctaaccttaaaacaacgtga
caaatgccagacaggacccatcagccaggcactgtgagagcacagagcagggaggttggg
tcctgcctgaggagacctggaagggaggcctcacaggaggatgaccaggtctcagtcagc
ggggaggtggaaagtgcaggtgcatcaggggcaccctgaccgaggaaacagctgccagag
gcctccactgctaaagtccacataaggctgaggtcagtcaccctaaacaacctgctccct
ctaagccaggggatgaGottggagcatcccacaagttccctaaaagttgcagcccccagg
gggattttgagctatcatctctgcacatgcttagtgagaagactacacaacatttctaag
aatctgagattttatattgtcagttaaccactttcattattcattcacctcaggacatgc
agaaatatttcagtcagaactgggaaacagaaggacctacattctgctgtcacttatgtg
tcaagaagcagatgatcgatgaggcaggtcagttgtaagtgagtcacattgtagcattaa
attctagtatttttgtagtttgaaacagtaacttaataaaagagcaaaagctaaaaaaaa
aaaaaaaaa

By “Epidermal Growth Factor Receptor (EGFR) polypeptide” is meant a polypeptide or fragment thereof having at least about 85% amino acid identity to NCBI Accession No. NP 005219.2 and having Epidermal Growth Factor (EGF) binding activity, as reproduced below (SEQ ID NO:4):

>NP_005219.2
MRPSGIAGAALLALLAALCPASRALEEKKVCQGTSNKLTQLGTFEDHFLSLQRMFNNCEV
VLGNLEITYVQRNYDLSFLKTIQEVAGYVLIALNTVERIPLENLQIIRGNMYYENSYALA
VLSNYDANKTGLKELPMRNLQEILHGAVRFSNNPALCNVESIQWRDIVSSDFLSNMSMDF
QNHLGSCQKCDPSCPNGSCWGAGEENCQKLEKIICAQQCSGRCRGKSPSDCCHNQCAAGC
TGPRESDCLVCRKFRDEATCKDTCPPLMLYNPTTYQMDVNPEGKYSFGATCVKKCPRNYV
VTDHGSCVRACGADSYEMEEDGVRKCKKCEGPCRKVCNGIGIGEFKDSLSINATNIKHFK
NCTSISGIDHILPVAFRGDSFTHTPPLDPQELDILKTVKEITGFLLIQAWPENREDLHAF
ENLEIIRGRTKQHGQFSLAVVSLNITSLGLRSLKEISDGDVIISGNKNLCYANTINWKKL
FGTSGQKTKIISNRGENSCKATGQVCHALCSPEGCWGPEPRDCVSCRNVSRGRECVDKCN
LLEGEPREFVENSECIQCHPECLPQAMNITCTGRGPDNCIQCAHYIDGPHCVKTCPAGVM
GENNTLVWKYADAGHVCHLCHPNCTYGCTGPGLEGCPTNGPKIPSIATGMVGALLLLLVV
ALGIGLFMRRRHIVRKRTIRRILQERELVEPLTPSGEAPNQALLRILKETEEKKIKVLGS
GAFGTVYKGLWIPEGEKVKIPVAIKELREATSPKANKEILDEAYVMASVDNPHVCRLLGI
CLISTVQLITQLMPFGCLLDYVREHKDNIGSQYLLNWCVQIAKGMNYLEDRRLVHRDLAA
RNVLVKTPQHVKITDFGLAKLLGAEEKEYHAEGGKVPIKWMALESILHRIYTHQSDVWSY
GVTVWELMTFGSKPYDGIPASEISSILEKGERLPQPPICTIDVYMIMVKCWMIDADSRPK
FRELIIEFSKMLRDPQRYLVIQGDERMHLPSPTDSNFYRALMDEEDMDDVVDADEYLIPQ
QGFFSSPSTSRTPLLSSLSATSNNSTVACIDRNGLQSCPIKEDSFLQRYSSDPEGALTED
SIDDTFLPVPEYINQSVPKRPAGSVQNPVYHNQPLNPAPSRDPHYQDPHSTAVGNPEYLN
TVQPTCVNSTFDSPAHWAQKGSHQISLDNPDYQQDFFPKEAKPNGIFKGSTAENAEYLRV
APQSSEFIGA

By “Epidermal Growth Factor (EGF) nucleic acid molecule” is meant a polynucleotide encoding an EGF polypeptide. An exemplary EGF nucleic acid molecule is provided at NCBI Accession No. NM_001963.5, and reproduced below (SEQ ID NO:5):

>NP_001963.5
aaaaagagaaactgttgggagaggaatcgtatctccatatttcttctttcagccccaatc
caagggttgtagctggaactttccatcagttcttcctttctttttcctctctaagccttt
gccttgctctgtcacagtgaagtcagccagagcagggctgttaaactctgtgaaatttgt
cataagggtgtcaggtatttcttactggcttccaaagaaacatagataaagaaatctttc
ctgtggcttcccttggcaggctgcattcagaaggtctctcagttgaagaaagagcttgga
ggacaacagcacaacaggagagtaaaagatgccccagggctgaggcctccgctcaggcag
ccgcatctggggtcaatcatactcaccttgcccgggccatgctccagcaaaatcaagctg
ttttcttttgaaagttcaaactcatcaagattatgctgctcactcttatcattctgttgc
cagtagtttcaaaatttagttttgttagtctctcagcaccgcagcactggagctgtcctg
aaggtactctcgcaggaaatgggaattctacttgtgtgggtcctgcacccttcttaattt
tctcccatggaaatagtatctttaggattgacacagaaggaaccaattatgagcaattgg
tggtggatgctggtgtctcagtgatcatggattttcattataatgagaaaagaatctatt
gggtggatttagaaagacaacttttgcaaagagtttttctgaatgggtcaaggcaagaga
gagtatgtaatatagagaaaaatgtttctggaatggcaataaattggataaatgaagaag
ttatttggtcaaatcaacaggaaggaatcattacagtaacagatatgaaaggaaataatt
cccacattcttttaagtgctttaaaatatcctgcaaatgtagcagttgatccagtagaaa
ggtttatattttggtcttcagaggtggctggaagcctttatagagcagatctcgatggtg
tgggagtgaaggctctgttggagacatcagagaaaataacagctgtgtcattcmatgtgc
ttgataagcggctgttttggattcagtacaacagagaaggaagcaattctcttatttgct
cctgtgattatgatggaggttctgtccacattagtaaacatccaacacagcataatttgt
ttgcaatgtccctttttggtgaccgtatcttctattcaacatggaaaatgaagacaattt
ggatagccaacaaacacactggaaaggacatggttagaattaacctccattcatcatttg
taccacttggtgaactGaaagtagtgcatccacttGcacaacccaaggcagaagatgaca
ottcmgagcctgagcagaaactttgcaaattgaggaaaggaaactgcagcagcactgtgt
gtgggcaagacctccagtcacacttgtgcatgtgtgcagagggatacgccctaagtcgag
accggaagtactgtgaagatgttaatgaatgtgctttttggaatcatggctgtactcttg
ggtgtaaaaacacccctggatcctattactgcacgtgccctgtaggatttgttctgcttc
otgatgggaaacgatgtcatcaacttgtttcctgtccacgcaatgtgtctgaatgcagcc
atgactgtgttctgacatcagaaggtcccttatgtttctgtcctgaaggctcagtgcttg
agagagatgggaaaacatgtagcggttgttcctcacccgataatggtggatgtagccagc
tctgcgttcctcttagcccagtatcctgggaatgtgattgctttcctgggtatgacctac
aactggatgaaaaaagctgtgcagcttcaggaccacaaccatttttgctgtttgccaatt
gtcaagatattcgacacatgcattttgatggaacagactatggaactctgctcagccagc
agatgggaatggtttatgccctagatcatgaccctgtcmaaaataagatatactttgccc
atacagccctgaagtggatagagagagctaatatggatggttcccagcgagaaaggctta
ttgaggaaggagtagatgtgccagaaggtcttgctgtggactggattggccgtagattct
attggacagacagagggaaatctctgattggaaGgagtgatttaaatgggaaacgttcca
aaataatcactaaggagaacatctctcaaccacgaggaattgctgttcatccaatggcca
agagattattctggactgatacagggattaatccaagaattgaaagttcttccctccaag
gccttggccgtctggttatagccagctctgatctaatctggcccagtggaataacgattg
acttcttaactgacaagttgtactggtgcgatgccaagcagtctgtgattgaaatgg0ca
atctggatggttcaaaacgccgaagacttacccagaatgatgtaggtcacccatttgctg
tagcagtgtttgagGattatgtgtggttctcagattgcmotatgccatcagtaatgagag
taaacaagaggactggcaaagatagagtacgtctccaaggcagcatgctgaagccctcat
cactggttgtggttcatccattggcaaaaccaggagcagatccctgcttatatcaaaacg
gaggctgtgaacatatttgcaaaaagaggcttggaactgcttggtgttcgtgtcgtgaag
gttttatgaaagcatcagatgggaaaacgtgtctggctctggatggtcatcagctgttgg
caggtggtgaagttgatctaaagaaccaagtaacaccattggacatcttgtccaagacta
gagtgtcagaagataacattacagaatctcaacacatgctagtggctgaaatcatggtgt
cagatcaagatgactgtgctcctgtgggatgcagcatgtatgctcggtgtatttcagagg
gagaggatgccacatgtcagtgtttgaaaggatttgctggggatggaaaactatgttctg
atatagatgaatgtgagatgggtgtcccagtgtgcccccctgcctcctccaagtgcatca
acaccgaaggtggttatgtctgccggtgctcagaaggctaccaaggagatgggattcact
gtcttgatattgatgagtgccaactgggggagcacagctgtggagagaatgccagctgca
caaatacagagggaggctatacctgcatgtgtgctggacgcctgtctgaaccagGactga
tttgccctgactctactccaccccctcacctcacggaagatgaccaccactattccgtaa
gaaatagtgactctgaatgtcccctgtcccacgatgggtactgcctccatgatggtgtgt
gcatgtatattgaagcattggacaagtatgcatgcaactgtgttgttggctacatagggg
agcgatgtcagtaccgagacctgaagtggtgggaactgcgccacgctggccacgggcagc
agcagaaggtcatcgtggtggctgtctgcgtggtggtgcttgtcatgctgctcctcctga
gcctgtggggggcccactactacaggactcagaagctgctatcgaaaaacccaaagaatc
cttatgaggagtcgagcagagatgtgaggagtcgcaggcctgctgacactgaggatggga
tgtcctcttgccctcaaccttggtttgtggttataaaagaacaccaagacctcaagaatg
ggggtcaaccagtggctggtgaggatggccaggcagcagatgggtcaatgcaaccaactt
catggaggcaggagccccagttatgtggaatgggcacagagcaaggctgctggattccag
tatccagtgataagggotcctgtccccaggtaatggagcgaagctttcatatgccctcct
atgggacacagacccttgaagggggtgtcgagaagccccattctctcctatcagctaacc
cattatggcaacaaagggccctggacccaccacaccaaatggagctgactcagtgaaaac
tggaattaaaaggaaagtcaagaagaatgaactatgt0gatgcacagtatcttttctttc
aaaagtagagcaaaactataggttttggttccacaatctctacgactaatcacctactca
atgcctggagacagatacgtagttgtgcttttgtttgctcttttaagcagtctcactgca
gtcttatttccaagtaagagtactgggagaatcactaggtaacttattagaaacccaaat
tgggacaacagtgctttgtaaattgtgttgtcttcagcagtcaatacaaatagatttttg
tttttgttgttcctgcagccccagaagaaattaggggttaaagcagacagtcacactggt
ttggtcagttacaaagtaatttctttgatctggacagaacatttatatcagtttcatgaa
atgattggaatattacaataccgttaagatacagtgtaggcatttaactcctcattggcg
tggtccatgctgatgattttgcaaaatgagttgtgatgaatcaatgaaaaatgtaattta
gaaactgatttcttcagaattagatggcttattttttaaaatatttgaatgaaaacattt
tatttttaaaatattacacaggaggcttcggagtttcttagtcattactgtccttttccc
ctacagaattttccctcttggtgtgattgcacagaatttgtatgtattttcagttacaag
attgtaagtaaattgcctgatttgttttcattatagacaacgatgaatttattctaatta
tttaaataaaatcaccaaaaacataaacattttattgtatgcctgattaagtagttaatt
atagtctaaggcagtactagagttgaaccaaaatgatttgtcaagcttgctgatgtttct
gtttttcgttttttttttttttccggagagaggataggatctcactctgttatccaggct
ggagtgtgcaatggcacaatcatagctcagtgcagcctcaaactcctgggctcaagcaat
cctcctgcctcagcctcccgagtaactaggaccacaggcacaggccaccatgcctggcta
aggtttttattttLattttttgtagacatggggatcacacaatgttgcccaggctggtct
tgaactcctggcctcaagcaaggtcgtgctggtaattttgcaaaatgaattgtgattgac
tttcagcctcccaacgtattagattataggcattagccatggtgcccagccttgtaactt
ttaaaaaaattttttagtctacaactctgtagattaaaatttcacatggtgttctaatta
aatatttttcttgcagccaagatattgttactacagataacacaacctgatatggtaact
ttaaattttgggggctttgaatcattcagtttatgcattaactagtccctttgtttatct
ttcatttctcaaccccttGtactttggtgataccagacatcagaataaaaagaaattgaa
gtacctgttttcaaatggatactttataggaattttggtaaagatttggtgatgggagga
tgacttgaggtttgtggatattagttaattattcagtatgatacctcacccagctaattt

By “Epidermal Growth Factor (EGF) polypeptide” is meant a polypeptide or fragment thereof having at least about 85% amino acid identity to NCBI Accession No. NP 001954.2 and corresponding to a pre-pro-protein form of EGF that is processed to produce a 53 amino acid EGF molecule (shown in bold) and having EGFR binding activity, as reproduced below (SEQ ID NO:6):

>NP_001934.2
MLLTLIILLPVVSKFSFVSLSAPQHWSCPEGTLAGNGNSTCVGPAPFLIFSHGNSIFRID
TEGTNYEQLVVDAGVSVIMDFHYNEKRIYWVDLERQLLQRVFLNGSRQERVCNIEKNVSG
MAINWINEEVIWSNQQEGIITVTDMKGNNSHILLSALKYPANVAVDPVERFIFWSSEVAG
SLYRADLDGVGVKALLETSEKITAVSLDVLDKRLFWIQYNREGSNSLICSCDYDGGSVHI
SKHPTQHNLFAMSLFGDRIFYSTWKMKTIWIANKHTGKDMVRINLHSSFVPLGELKVVHP
LAQPKAEDDTWEPEQKLCKLRKGNCSSTVCGQDLQSHLCMCAEGYALSRDRKYCEDVNEC
AFWNHGCTLGCKNTPGSYYCTCPVGFVLLPDGKRCHQLVSCPRNVSECSHDCVLTSEGPL
CFCPEGSVLERDGKTCSGCSSPDNGGCSQLCVPLSPVSWECDCFPGYDLQLDEKSCAASG
PQPFLLFANSQDIRHMHFDGTDYGTLLSQQMGMVYALDHDPVENKTYFAHTALKWIERAN
MDGSQRERLIEECVDVPEGLAVDWIGRREYWTDRGKSLIGRSDLNGKRSKIITKENISQP
RGIAVHPMAKRLFWTDTGINPRIESSSLQGLGRLVIASSDLIWPSGITIDFLTDKLYWCD
AKQSVIEMANLDGSKRRRLTQNDVGHPFAVAVPEDYVWFSDWAMPSVMRVNKRTGKDRVR
LQGSMLKPSSLVVVHPLAKPGADPCLYQNGGCEHICKKRLGTAWCSCREGFMKASDGKTC
LALDGHQLLAGGEVDLKNQVTPLDILSKTRVSEDNITESQHMLVAEIMVSDQDDCAPVGC
SMYARCISEGEDATCQCLKGFAGDGKLCSDIDECEMGVPVCPPASSKCINTEGGYVCRCS
EGYQGDGIHCLDIDECQLGEHSCGENASCTNTEGGYTCMCAGRLSEPGLICPDSTPPPHL
REDDHHYSVRNSDSECPLSHDGYCLIMGVCMYIEALDKYACNCVVGYIGERCQYRDLKWW
ELRHAGHGQQQKVIVVAVCVVVLVMLLLLSLWGAHYYRTQKLLSKNPKNPYEESSRDVRS
RRPADTEDGMSSCPQPWFVVIKEHQDLKNGGQPVAGEDGQAADGSMQPTSWRQEPQLCGM
GTEQGCWIPVSSDKGSCPQVMERSFHMPSYGTQTLEGGVEKPHSLLSANPLWQQRALDPP
HQMELTQ

By “Neuregulin 1 (NRG1) nucleic acid molecule” is meant a polynucleotide encoding an NRG1 polypeptide. An exemplary NRG1 nucleic acid molecule is provided at NCBI Accession No. BC150609.1, and reproduced below (SEQ ID NO:7):

>BC150609.1
gagcccttggaccaaactcgcctgcgccgagagccgtccgcgtagagcgctccgtctccg
qcgagatgtccgagcgcaaagaaggcagaggcaaagggaagggcaagaagaaggagcgag
gctccggcaagaagccggagtccgcggcgggcagccagagcccagccttgcctccccaat
tgaaagagatgaaaagccaggaatcggctgcaggttccaaactagtccttcggtgtgaaa
ccagttctgaatactcctctctcagattcaagtggttcaagaatgggaatgaattgaatc
gaaaaaacaaaccacaaaatatcaagatacaaaaaaagccagggaagtcagaacttcgca
ttaacaaagcatcactggctgattctggagagtatatgtgcaaagtgatcagcaaattag
gaaatgacagtgcctctgccaatatcaccatcgtggaatcaaacgagatcatcactggta
tgccagcctcaactgaaggagcatatgtgtcttcagagtctcccattagaatatcagtat
ccacagaaggagcaaatacttcttcatctacatctacatccaccactgggacaagccatc
ttgtaaaatgtgcggagaaggagaaaactttctgtgtgaatggaggggagtgcttcatgg
tgaaagacctttcaaacccctcgagatacttgtgcaagtgccaacctggattcactggag
caagatgtactgagaatgtgcccatgaaagtccaaaaccaagaaaaggcggaggagctgt
accagaagagagtgctgaccataaccggcatctgcatcgccctccttgtggtcggcatca
tgtgtttggtggcctactgcaaaaccaagaaacagcGgaaaaagctgcatgaccgtcttc
qgcagagccttcggtctgaacgaaacaatatgatgaacattgccaatgggcctcaccatc
ctaacccaccccccgagaatgtccagctggtgaatcaatacgtatctaaaaacgtcatct
ccagtgagcatattgttGagagagaagcagagacatccttttccaccagtcactatactt
ccacagcccatcactccactactgtcacccagactcctagccacagctggagcaacggac
acactgaaagcatcctttccgaaagccactctgtaatcgtgatgtcatccgtagaaaaca
gtaggcacagcagcccaactgggggcccaagaggacGtcttaatggcacaggaggccctc
gtgaatgtaacagcttcctcaggcatgccagagaaacccctgattcctaccgagactctc
ctcatagtgaaaggtatgtgtcagccatgaccaccccggctcgtatgtcacctgtagatt
tccacacgccaagctcccccaaatcgcccccttoGgaaatgtctccacccgtgtccagca
tgacggtgtccatgccttccatggcggtcagccccttcatggaagaagagagacctctac
ttctcgtgacaccaccaaggctgcgggagaagaagtttgaccatcaccctcagcagttca
gctccttccaccacaaccccgcgcatgacagtaacagcctccctgctagccccttgagga
tagtggaggatgaggagtatgaaacgacccaagagtacgagccagcccaagagcctgtta
agaaactcgccaatagccggcgggccaaaagaaccaagcccaatggccacattgctaaca
gattggaagtggacaGcaacacaagctcccagagcaGtaactcagagagtgaaacagaag
atgaaagagtaggtgaagatacgcctttcctgggcatacagaaccccctggcagccagto
ttgaggcaacacctgccttccgcctggctgacagcaggactaacccagcaggccgcttct
cgacacaggaagaaatccaggccaggctgtctagtgtaattgctaaccaagaccctattg
otgtataaaacctaaataaacacatagattcacctgtaaaactttattttatataataaa
gtattccaccttaaattaaacaatttattttattttagcagttctgcaaatagaaaacag
gaaaaa

By “Neuregulin 1 (NRG1) polypeptide” is meant a polypeptide or fragment thereof having at least about 85% amino acid identity to NCBI Accession No. AAI50610.1 and having Neuregulin 1 (NRG1) binding activity, as reproduced below (SEQ ID NO:8):

>AAI50610.1
MSERKEGRGKGKGKKKERGSGKKPESAAGSQSPALPPQLKEMKSQESAAG
SKIVERCETSSEYSSLREKWFKNGNELNRKNKPQNIKIQKKPGKSELRIN
KASLADSGEYMCKVISKLGNDSASANITIVESNEIITGMPASTEGAYVSS
ESPIRISVSTEGANTSSSTSTSTIGTSHLVKCAEKEKTFCVNGGECFMVK
DLSNPSRYLCKCQPGFTGARCTENVPMKVQNQEKAEELYQKRVLTITGIC
IALLVVGIMCLVAYCKTKKQRKKLHDRLRQSLRSERNNMMNIANGPHHPN
PPPENVQLVNQYVSKNVISSEHIVEREAETSFSTSHYTSTAHHSTTVTQT
PSHSWSNGHTESILSESHSVIVMSSVENSRHSSPTGGPRGPLNGTGGPRE
CNSFLRHARETPDSYRDSPHSERYVSAMTTPARMSPVDFHTPSSPKSPPS
EMSPPVSSMTVSMPSMAVSPFMEEERPLLLVTPPRLREKKFDHHPQQFSS
FHHNPAHDSNSLPASPLRIVEDEEYETTQEYEPAQEPVKKLANSRRAKRT
KPNGHIANRLEVDSNTSSQSSNSESETEDERVGEDTPFLGIQNPLAASLE
ATPAFRLADSRTNPAGRFSTQEEIQARLSSVIANQDPIAV

By “Neuregulin 1β (NRG1β) nucleic acid molecule” is meant a polynucleotide encoding an NRG1 polypeptide. An exemplary NRG1β nucleic acid molecule is provided at NCBI Accession No. NM_001322205.1 and reproduced below (SEQ ID NO:9):

>NM_001322205.1
ggcttaactgatgcctgcctgcctctctttgatttgatggcctttattccttctaattgg
ataaaataggaagtcactggcagtcctgtgtggctggggatactgattttactcagacca
gcctgcagctctagagtgtgggtagagagcggggagtgggggttgggagagggggaggaa
agagagagaggagagaggacgggcttggatgaagaatggaaagaaagagaaagagactga
agcagagaagagccgcagaggaagaaagtgaatgagcactcaagaaggacaaagaggagt
agtcgggggtggggtggaggcagggcggggaagggagtgaccgcccctcctggctgcact
cttgcctccggagccctctgatcctgtttgcagtgatgctccgagggcaggcacctgctg
ctctgtaatgattcagcccctttcagccgtcgtcgcgttaacacaacaggatgctgttgc
tattgtcactactgcctctcctgccgccgctgctgctgccgccgccgccacagccgctgg
tcctccttctgcttttacttctcctgcatgacagttgttttcttcatctgagcagacacc
agcttcagatgctcgaggtgagaaacatgcctttcagtttgggctactggtttacttaat
taatcagccggcagctcagtcgatctattttcgtccctgtcctcttgacgagcccgggat
ggtttggagtagcatttaaaagaactagaaaagtggcccagaaacagcagcttaaagaat
tattacgatatactttgattttgtagttgctaggagcttttcttccccccttgcatcttt
ctgaactcttcttgattttaataatggccttggacttggacgatttatcgatttccccct
gtaagatgctgtatcatttggttgggggggcctctgcgtggtaatggaccgtgagagcgg
ccaggccttcttctggaggtgagccgatggagatttattccccagacatgtctgaggtcg
ccgccgagaggtcctccagcccctccactcagctgagtgcagacccatctcttgatgggc
ttccggcagcagaagacatgccagagccccagactgaagatgggagaacccctggactcg
tgggcctggccgtgccctgctgtgcgtgcctagaagctgagcgcctgagaggttgcctca
actcagagaaaatctgcattgtccccatcctggcttgcctggtcagcctctgccccagca
tcgccggcctcaagtgggtatttgtggacaagatctttgaatatgactctcctactcacc
ttgaccctggggggttaggccaggaccctattatttctctggacgcaactgctgcctcag
ctgtgtgggtgtcgtctgaggcatacacttcacctgtctctagggctcaatctgaaagtg
aggttcaagttacagtgcaaggtgacaaggctgttgtctcctttgaaccatcagcggcac
cgacaccgaagaatcgtatttttgccttttctttcttgccgtccactgcgccatccttcc
cttcacccacccggaaccctgaggtgagaacgcccaagtcagcaactcagccacaaacaa
cagaaactaatctccaaactgctcctaaactttctacatctacatccaccactgggacaa
gccatcttgtaaaatgtgcggagaaggagdaaactttctgtgtgaatggaggggagtgct
tcatggtgaaagacctttcaaacccctcgagatacttgtgcaagtgcccaaatgagttta
ctggtgatcgctgccaaaactacgtaatggccagcttctacaagcatcttgggattgaat
ttatggaggcggaggagctgtaccagaagagagtgctgaccataaccggcatctgcatcg
ccctccttgtggtggcatcatgtgtqtggtggcctactgcaaaaccaactaaacagcgga
aaaagctgcatgaccgtcttcggcagagccttcggtctgaacgaaacaatatgatgaaca
ttgccaatgggcctcaccatcctaacccaccccccgagaatgtccagctggtgaatcaat
acgtatctaaaaacgtcatctccagtgagcatattgttgagagagaagcagagacatcct
tttccaccagtcactatacttccacagcccatcactccactactgtcacccagactccta
gccacagctggagcaacggacacactgaaagcatcctttccgaaagccactctgaaatcg
tgatgtcatccgtagaaaacagtaggcacagcagcccaactgggggcccaagaggacgtc
ttaatggcacaggaggccctcgtgaatgtaacagcttcctcaggcatgccagagaaaccc
ctgattcctaccgagactctcctcatagtgaaaggtatgtgtcagccatgaccaccccgg
ctcgtatgtcacctgtagatttccacacgccaagctcccccaaatcgcccccttaggaaa
tgtctccacccgtgtccagcatgacggtgtccatgccttccatggcggtcagccccttca
tggaagaagagagacctctacttctcgtgacaccaccaaggctgcgggagaagaagtttg
accatcaccctcagcagttcagctccttccaccacaaccccgcgcatgacagtaacagcc
tccctgctagccccttgaggatagtggaggatgaggagtatgaaacgacccaagagtacg
agccagcccaagagcctgttaagaaactcgccaatagccggcgggccaaaagaaccaagc
ccaatggccacattgctaacagattggaagtggacagcaacacaagctcccagagcagta
actcagagagtgaaacagaagatgaaagagtaggtgaagatacgcctttcctgggcatac
agaaccccctggcagccagtcttgaggcaacacctgccttccgcctggctgacagcagga
ctaacccagcaggcagcttctcgacacaggaagaaatccaggccaggctgtctagtgtaa
ttgctaaccaagaccctattgctgtataaaacctaaataaacacatagattcacctgtaa
aactttattttatataatatagtattccaccttaaattaaacaatttattttattttagc
agttctgcaaatagaaaacaggaaaaaaacttttataaattaaatatatgtatgtaaaaa
tgtgttatgtgccatatgtagcaattttttacagtatttcaaaacgagaaagatatcaat
ggtgcctttatgttatgttatgtcgagagcaagttttgtacagttacagtgattgctttt
ccacagtatttctgcaaaacctctcatagattcagtttttgctggcttcttgtgcattgc
attatgatgttgactggatgtatgatttgcaagacttacaactgtccctctgtttgcttg
tagtagcacccgatcagtatgtcttgtaatggcacatccatccagatatgcctctcttgt
gtatgaagttttctttgctttcagaatatgaaatgagttgtgtctactctgccagccaaa
ggtttgcctcattgggctctgagataatagtagatccaacagcatgctactattaaatac
agcaagaaactgcattaagtaatgttaaatattaggaagaaagtaatactgtgatttaaa
aaaaactatattattaatcagaagacagcttgctcttactaaaaggagctctcatttact
ttatttgattttatttttcttgacaaaaagcaacagttttagggatagcttagaaaatgg
gttctggcttgctatcagggtaaatctaacaccttacaagaggactgagtgtcactttct
ctctgggggaatgatccagcagcttatctagttgacaatcaaaacacggctgataaaggt
gcaatcatttctgacatgtatttttcactgattttgaagctagtgattggttgtgtcttc
ttggctcaaaaagaagcatattacggcacaaaaagcccagcccagacagcacatgcagca
ttttgtctgaaatacttctagagtcaaacgtgcctgctgtacatagcgatgacttgtcat
catagggaagtatttccatcgtagagtgttcagaaggagtgactgtataggtggagagaa
gcttagtgactccgttgaaattttaaaatgtggatgaccacccctttctcccccttattt
ttcttttatctttccatgttgccttgatcaggtcataactatgcatgaacattttttatc
aggaatggccgatgtgtatgtgatttgtaatcacaagtaatgattcatcaggaaatgtca
atcctgttggaaagattgcacctttacttgcagaagtgacccccacctgtgtcctgacct
ctccatttacaggctctctcacccatttcccccacctcctttaatttttgctttactgtc
ataaagtaggactaagattggtctaagcattgcatgttcttttgtgatggtaaatccaaa
ggaaggcctataagtattaacatttgaaataactgctaattcaggaaaatggaagaaaaa
aaattatttgaaacacagaacccatttcatggcctgcctgatatctgtgaaatcagggct
ggagctttacttaggattcacatggcctcctaggaaccatgggacaaatgggaaacaggt
tatcgggggattcatgaagtcagtgagagtaattgcttcttttttgcgggtgaactgaat
gtatttcttcaccaaatcttgatgttaacaattaaaaagaagaaatgacatgcaagtagg
tcttagcagaaaaatgcaggctgggcatgagtcatgttgttaccctcccacatgctccta
caatccacagagatgcctgtctgcaggttcttgaagttattgttagtatttggtatctca
aatttttcgtcactgttcacatgccactttctctgtgcacagtggtatcctcatttgctt
tttaacctacactgaggagtctttgtcaggttgcactgattttccaattctgcagtaatg
agtaagctcacggcatggggaagaagacagtcagtccaatgaagttctctaaattatttt
aacattgcctttgaaggccttgactcatccttagctatttcaatgaagaaattcctacca
tgaatttaaaaccctaaaaattctgtttcaaattctttgggcattggggtactcagatat
cccattgtggaagaattttaagaataaatagaagtttctgttgagaaccatgagcaacat
gtttcttacaatgagaattgctatgcattttaaaattgcaaatatatatgaaaattgaag
acaagaggaaattgtatttctaacttgattctgatcactcacagaggtggcatattatta
tagttgggacatcctttgcacccttcataaaaaaggccagctgactgctcagcatcacct
gccaaggccactagatttgtgtttacaggggtatctctgtgatgcttgtcacatcactct
tgaccacctctgttaataaattccgacagtgcagtggcgatcggagtgtgaacttatgtt
cccagcatatggaaagctatcttaggttttaaggtagtagaaattgcccaggagtttgac
agcaactttgtttcccgggtctaaaatcgtatcccactgaggtgtatgcagtggagcata
atacatgcaaatacatgcaaaactccttttgtttcacctaagattcactttctatcttac
tttcccttcctgcctagtgtgacttttgcccccaagagtgcctggacagcattctagttt
ctacaaaatggtcctctgtgtaggtgaatgtgtcccaaacctgctatcactttcttgttt
cagtgtgactgtcttgttagaggtgaagtttatccagggtaacttgctcactaactattc
ctttttatggcctggggttaaagggcgcatggctcacactggtgaaaataaggaaggcct
ggtcttatcttgtattaataatactggctgcattccaccagccagagatttctatctgcg
aagacctatgaaacactgaagagaaatgtaggcagaaggaaatggccacatatcacaagt
tctattatatattcttttgtaaatacatattgtatattacttggatgttttcttatatca
tttactgtctttttgagttaatgtcagtttttactctctcaacttactatgtaacattgt
aaataacataatgtcctttattatttatatttaagcatctaacatatagagttgttttca
tataagtttaagataaatgtcaaaaatatatgttcttttgtttttctttgctttaaaatt
atgtatcttttccttttcttttttttaagaataatttattgttcaggagaaagaatgtat
atgtaactgaaactatctgaagaatgcacattgaaggccgtgaggtactgataaactaaa
gaatttattattcaaaatactaagcaataagtaattgtgatttatttaaagttttgtcca
ttttccatgaaagacatactgcaataaaaatgctactctgtggaaaaaaaaaaaaaaaaa
a

By “Neuregulin 1β (NRG11) polypeptide” is meant a polypeptide or fragment thereof having at least about 85% amino acid identity to NCBI Accession No. NP_001309134.1 and having Neuregulin 1 (NRG1) binding activity, as reproduced below (SEQ ID NO:10):

>NP_001309134.1
MEIYSPDMSEVAAERSSSPSTQLSADPSLDGLPAAEDMPEPQTEDGRTPG
LVGLAVPCCACLEAERLRGCLNSEKICIVPILACLVSLCLCIAGLKWVEV
DKIFEYDSPTHLDPGGLGQDPIISLDATAASAVWVSSEAYTSPVSRAQSE
SEVQVTVQGDKAVVSFEPSAAPTPKNRIFAFSFLPSTAPSFPSPTRNPEV
RTPKSATQPQTTETNLQTAPKLSTSTSTIGISHINKCAEKEKTFCVNGGE
CFMVKDLSNPSRYLCKCPNEFTGDRCQNYVMASFYKHLGIEFMEAEELYQ
KRVLILTGICIALINVGIMCVVAYCKTKKQRKEIHDPIRQSLRSERNNMM
NIANGPHHPNPPPENVQLVNQYVSKNVISSEHIVEREAETSFSTSHYTST
AHHSTTVTQTPSHSWSNGHTESILSESHSVIVMSSVENSRHSSPIGGPRG
RLNGIGGPRECNSFLRHARETPDSYRDSPHSERYVSAMTTPARMSPVDFH
TPSSPKSPPSEMSPPVSSMTVSMPSMAVSPFMEHERPLLLVIPPRLREKK
EDHHPQOFSSFHHNPAHDSNSLPASPLRIVEDEEYETTQEYEPACEPVKK
LANSRRAKRTKPNGHIANRLEVDSNISSQSSNSESETEDERVGEDTPFLG
IQNPLAASLEATPAFRLADSRTNPAGRFSTEIQARLSSVIANQDPIAV

By “NRG-BVN hybrid polypeptide” is meant a polypeptide or fragment thereof having at least about 85%, 90%, 91%, 92%, 93%, 94%, 95%, 96%, 97%, 98%, or 99% amino acid identity to the amino acid sequence below (SEQ ID NO:11):

>NRG-BVN-hybrid
GTSHLVKCPLSHEAYCVNGGECFMVKDLSNPSRYLCKCPNEFTGERCQNY
VMASF

By “TGFα hybrid polypeptide” is meant a polypeptide or fragment thereof having at least about 85%, 90%, 91%, 92%, 93%, 94%, 95%, 96%, 97%, 98%, or 99% amino acid identity to the amino acid sequence below (SEQ ID NO:12):

>TGF-BVN-hybrid
NTENDCPLSHEAYCLHDGVCRFLVQEDKPACVCVVGYVGERCQFRDLRWW
DAP

By “initial IN-02 polypeptide” is meant or fragment thereof having at least about 85%, 90%, 91%, 92%, 93%, 94%, 95%, 96%, 97%, 98%, or 99% amino acid identity to the amino acid sequence below (SEQ ID NO:13):

>Initial_IN-02_polypeptide
MTPQNITDLCAEYHNTQIHTLNDHIFSYTESLAGKREMAIITFKNGATFQ
VEVPGSQHIDSQKKAIERMKDTLRIAYLTEAKVEKLCVWNNKTPHAIAAI
SMANSSGGSGGGSGTFYDIETLKVIDEEWQRTQCHPIETLVDIFQEYPDE
IFYIFKPSCVPLMRCGGCCNDEG

By “processed or final IN-02 polypeptide” is meant or fragment thereof having at least about 85%, 90%, 91%, 92%, 93%, 94%, 95%, 96%, 97%, 98%, or 99% amino acid identity to the amino acid sequence below (SEQ ID NO:14):

>Final_IN-02_polypeptide
TPQNITDLCAEYHNTQIHTLNDKIFSYTESTAGKREMAIITFKNGATFQV
EVPGSQHIDSQKKAIERMKDTLRIAYLTEAKVEKLCVNNNKTPHAIAAIS
MANSSGGSGGGSGTFYDETLKVIDEEWQRTQCHPIETLVDIFQEYPDEIE
YIFKPSCVPLMRCGGCCNDEG

By “vascular endothelial growth factor A (VEGF-A) nucleic acid molecule” is meant a polynucleotide encoding a VEGF-A polypeptide. An exemplary VEGF-A nucleic acid molecule is provided at NCBI Accession No. NM_001025366.3 and reproduced below (SEQ ID NO:15):

>NM_001025366.3
gcggaggcttggggcagccgggtagctcggaggtcgtggcgctgggggctagcaccagcg
ctctgtcgggaggcgcagcggttaggtggaccggtcagcggactcaccggccagggcgct
cggtgctggaatttgatattcattgatccgggttttatccctcttcttttttcttaaaca
tttttttttaaaactgtattgtttctcgttttaatttatttttgcttgccattccccact
tgaatcgggccgacggcttggggagattgctctacttccccaaatcactgtggattttgg
aaaccagcagaaagaggaaagaggtagcaagagctccagagagaagtcgaggaagagaga
gacggggtcagagagagcgcgcgggcgtgcgaccagcgaaagcgacaggggcaaagtgag
tgacctgcttttgggggtgaccgccggagcgcggcgtgagccctcccccttgggatcccg
cagctgaccagtcgcgctgacggacagacagacagacaccgcccccagccccagctacca
cctcctccccggccggcggcggacagtggacgcggcggcgagccgcgggcaggggccgga
gcccgcgcccggaggcggggtggagggggtcggggctcgcggcgtcgcactgaaactttt
cgtccaacttctgggctgttctcgcttcggaggagccgtggtccgcgcgggggaagccga
gccgagcggagccgagagaagtgctagctcgggccgggaggagccgcagccggaggaggg
ggaggaggaagaagagaaggaagaggagagggggccgcagtggcgactcggcgctcggaa
gccgggctcatggacgggtgaggcggcggtgtgcgcagacagtgctccagccgcgcgcgc
tccccaggccctggccagggcctcgggccggggaggaagagtagctcgccgaggagccga
ggagagcgggccgccccacagcccgagccggagagggagcgcgagccgcgccggccccgg
tcgggcctccgaaaccatgaactttctgctgtcttgggtgcattggagccttgccttgct
gctctacctccaccatgccaagtggtcccaggctgcacccatggcagaaggaggagggca
gaatcatcacgaagtggtgaagttcatggatgtctatcagcgcagctactgccatccaat
cgagaccctggtggacatcttccaggagtaccctgatgagatcgagtacatcttcaagcc
atcctgtqtgcccctgatgcgatgcgggggctgctgcaatgacgagggcctggagtgtgt
gcccactgaggagtccaacatcaccatgcagattatgcggatcaaacctcaccaaggcca
gcacataggagagatgagcttcctacagcacaacaaatgtgaatgcagaccaaagaaaga
tagagcaagacaagaaaaaaaatcagttcgaggaaagggaaaggggcaaaaaagaaagcg
caagaaatcccggtataaGtcctggagcgtgtacgttggtgcccgctgctgtctaatgcc
ctggagcctccctggcccccatccctgtgggccttgctcagagcggagaaagcatttgtt
tgtacaagatccgcagacgtgtaaatgttcctgcaaaaacacagactcgcgttgcaaggc
gaggcagcttgagttaaacgaacgtacttgcagatgtGacaagccgaggcggtgagccgg
gcaggaggaaggagcctccctcagggtttcgggaaccagatctctcaccaggaaagactg
atacagaacgatcgatacagaaaccacgctgccgccaccacaccatcaccatcgacagaa
cagtccttaatccagaaacctgaaatgaaggaagaggagactctgcgcagagcactttgg
gtccggagggcgagactccggcggaagcattcccgggcgggtgacccagcacggtccctc
ttggaattggattcgccattttatttttcttgctgctaaatcaccgagcccggaagatta
gagagttttatttctgggattcctgtagacacacccacccacatacatacatttatatat
atatatattatatatatataaaaataaatatctctattttatatatataaaatatatata
ttctttttttaaattaacagtgctaatgttattggtgtcttcactggatgtatttgactg
ctgtggacttgagttgggaggggaatgttcccactcagatcctgacagggaagaggagga
gatgagagactctggcatgatcttttttttgtcccacttggtggggccagggtcctctcc
cctgcccaggaatgtgcaaggccagggcatgggggcaaatatgacccagttttgggaaca
ccgacaaacccagccctggcgctgagcctctctaccccaggtcagacggacagaaagaca
gatcacaggtacagggatgaggacaccggctctgaccaggagtttggggagcttcaggac
attgctgtgctttggggattccctccacatgctgcacgcgcatctcgcccccaggggcac
tgcctggaagattcaggagcctgggcggccttagcttactctcacctgcttctgagttgc
ccaggagaccactggcagatgtcccggcgaagagaagagacacattgttggaagaagcag
cccatgacagctccccttcctgggactcgccctcatcctcttcctgctccccttcctggg
gtgcagcctaaaaggacctatgtcctcacaccattgaaaccactagttctgtccccccag
gagacctggttgtgtgtgtgtgagtggttgaccttcctccatcccctggtccttcccttc
ccttcccgaggcacagagagacagggcaggatccacgtgcccattgtggaggcagagaaa
agagaaagtgttttatatacggtacttatttaatatccctttttaattagaaattaaaac
agttaatttaattaaagagtagggttttttttcagtattcttggttaatatttaatttca
actatttatgagatgtatcttttgctctctcttgctctcttatttgtaccggtttttgta
tataaaattcatgtttccaatctctctctccctgatcggtgacagtcactagcttatctt
gaacagatatttaattttgctaacactcagctctgccctccccgatcccctggctcccca
gcacacattcctttgaaataaggtttcaatatacatctacatactatatatatatttggc
aacttgtatttgtgtgtatatatatatatatatgtttatgtatatatgtgattctgataa
aatagacattgctattctgttttttatatgtaaaaacaaaacaagaaaaaatagagaatt
ctacatactaaatctctctccttttttaattttaatatttgttatcatttatttattggt
gctactgtttatccgtaataattgtggggaaaagatattaacatcacgtctttgtctcta
gtgcagtttttcgagatattccgtagtacatatttatttttaaacaacgacaaagaaata
cagatatatcttaaaaaaaaaaaagcattttgtattaaagaatttaattctgatctcaaa

By “VEGF-A polypeptide” is meant a polypeptide encoded by a VEGF-A nucleic acid molecule. An exemplary VEGF-A nucleic acid molecule is provided at NCBI Accession No. NP_001020537.2 and reproduced below (SEQ ID NO:16):

>NP_001020537.2
MTDRQTDTAPSPSYHLLPGRRRTVDAAASRGQGPEPAPGGGVEGVGARGV
ALKLFVQLLGCSRFGGAVVRAGEAEPSGAARSASSGREEPQPEEGEEEEE
KEEERGPQWRLGARKPGSWTGEAAVCADSAPAARAPQALARASGRGGRVA
RRGAEESGPPHSPSRRGSASRAGPGRASETMNFLLSWVHWSLALLLYLHH
AKWSQAAPMAEGGGQNHHEVVKFMDVYQRSYCHPIETLVDIFQEYPDEIE
YIFKPSCVPLMRCGGCCNDEGLECVPTEESNITMQIMRIKPHQGQHIGEM
SFLQHNKCECRPKKDRAROEKKSVRGKGKGQKRKRKKSRYKSWSVYVGAR
CCLMPWSLPGPHPCGPCSERRKHLFVQDPQTCKCSCKNTDSRCKARQLEL
NERTCRCDKPRR

By “vascular endothelial growth factor A (VEGF-D) nucleic acid molecule” is meant a polynucleotide encoding a VEGF-D polypeptide. An exemplary VEGF-D nucleic acid molecule is provided at NCBI Accession No. NM_004469 and reproduced below (SEQ ID NO:17):

>NM_004469
aagacacatgcttctgcaagcttccatgaaggttgtgcaaaaaagtttcaatccagagtt
gggttccagctttctgtagctgtaagcattggtggccacaccacctccttacaaagcaac
tagaacctgcggcatacattggagagatttttttaattttctggacatgaagtaaattta
gagtgctttctaatttcaggtagaagacatgtccaccttctgattatttttggagaacat
tttgatttttttcatctctctctccccacccctaagattgtgcaaaaaaagcgtaccttg
cctaattgaaataatttcattggattttgatcagaactgattatttggttttctgtqtga
agttttgaggtttcaaactttccttctggagaatgccttttgaaacaattttctctagct
gcctgatgtcaactgcttagtaatcagtggatattgaaatattcaaaatgtacagagagt
gggtagtggtgaatgttttcatgatgttgtacgtccagctggtgcagggctccagtaatg
aacatggaccagtgaagcgatcatctcagtccacattggaacgatctgaacagcagatca
gggctgcttctagtttggaggaactacttcgaattactcactctgaggactggaagctgt
ggagatgcaggctgaggctcaaaagttttaccagtatggactctcgctcagcatcccatc
ggtccactaggtttgcggcaactttctatgacattgaaacactaaaagttatagatgaag
aatggcaaagaactcagtgcagccctagagaaacgtgcgtggaggtggccagtgagctgg
ggaagagtaccaacacattcttcaagcccccttgtqtgaacgtgttccgatgtggtggct
gttgcaatgaagagagccttatctgtatgaacaccagcacctcgtacatttccaaacagc
tctttgagatatcagtgcctttgacatcagtacctgaattagtgcctgttaaagttgcca
atcatacaggttgtaagtgcttgccaacagccccccgccatccatactcaattatcagaa
gatccatccagatccccgaagaagatcgctgttcccattccaagaaactctgtcctattg
acatgctatgggatagcaacaaatgtaaatgtgttttgcaggaggaaaatccacttgctg
gaacagaagaccactctcatctccaggaaccagctctctgtgggccacacatgatgtttg
acgaagatcgttgcgagtgtgtctgtaaaacaccatgtcccaaagatctaatccagcacc
ccaaaaactgcagttgctttgagtgcaaagaaagtctggagacctgctgccagaagcaca
agctatttcacccagacacctgcagctgtgaggacagatgcccctttcataccagaccat
gtgcaagtggcaaaacagcatgtgcaaagcattgccgctttccaaaggagaaaagggctg
cccaggggccccacagccgaaagaatccttgattcagcgttccaagttccccatccctgt
catttttaacagcatgctgctttgccaagttgctgtcactgtttttttcccaggtgttaa
aaaaaaaatccattttacacagcaccacagtgaatccagaccaaccttccattcacacca
gctaaggagtccctggttcattgatggatgtcttctagctgcagatgcctctgcgcacca
aggaatggagaggaggggacccatgtaatccttttgtttagttttgtttttgttttttgg
tgaatgagaaaggtgtgctggtcatggaatggcaggtgtcatatgactgattactcagag
0agatgaggaaaactgtagtctctgagtcctttgctaatcgcaactcttgtgaattattc
tgattcttttttatgcagdatttgattcgtatgatcagtactgactttctgattactgtc
cagcttatagtcttccagtttaatgaactaccatctgatgtttcatatttaagtgtattt
aaagaaaataaacaccattattcaagcca

By “VEGF-D polypeptide” is meant a polypeptide encoded by a VEGF-D nucleic acid molecule. An exemplary VEGF-D nucleic acid molecule is provided at NCBI Accession No. NP_004460.1 and reproduced below (SEQ ID NO:18):

>NP_004460.1
MYREWVVVNVFMMLYVOLVQGSSNEHGPVKRSSQSTLERSEQQIRAASSLEEL
LRITHSEDWKLWRCRLRLKSFTSMDSRSASHRSTRFAATFYDIETLKVIDEEW
QRTQCSPRETCVEVASELGKSTNTFFKPPCVNVFRCGGCCNEESLICMNTSTS
YISKQLFEISVPLTSVPELVPVKVANHTGCKCLPTAPRHPYSIIRRSIQIPEE
DRCSHSKKLCPIDMLWDSNKCKCVLQEENPLAGTEDHSHLQEPALCGPHMMFD
EDRCECVCKTPCPKDLIQHPKNCSCFECKESLETCCQKHKLFHPDTCSCEDRC
PFHTRPCASGKTACAKHCRFPKEKRAAQGPHSRKNP

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it is understood that the particular value forms another aspect. It is further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. It is also understood that throughout the application, data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

Where applicable or not specifically disclaimed, any one of the embodiments described herein are contemplated to be able to combine with any other one or more embodiments, even though the embodiments are described under different aspects of the disclosure.

These and other embodiments are disclosed and/or encompassed by, the following detailed description.

### BRIEF DESCRIPTION OF THE DRAWINGS

The following detailed description, given by way of example, but not intended to limit the disclosure solely to the specific embodiments described, may best be understood in conjunction with the accompanying drawings, in which:

FIGS. 1A-1D depict two protein schematics, a recombinant protein sequence, and a bar graph, respectively. FIG. 1A is a ribbon diagram protein schematic that illustrates a chimeric VEGF molecule (VEGF-DA) according to an exemplary embodiment of the disclosure that includes the N-terminal region derived from VEGF-D and the ‘homology domain’ from VEGF-A (VEGFR-1, VEGFR-2, and VEGFR-3 binding regions are indicated.

FIG. 1B is a protein schematic illustrates the structure and organization of IN-02, in which a chimeric VEGF-DA protein domain is fused to the C-terminus of CTB via 10 amino acid linker according to an exemplary embodiment of the disclosure. FIG. 1C depicts the protein sequence of IN-02, color coordinated to match FIGS. 1A-1B. The initiating methionine residue is removed by methionine aminopeptidase, so is absent from the mature protein. FIG. 1D is a bar graph showing the effect of VEGF-A, VEGF-D, and IN-02, alone and in combination with neutralizing antibodies (NAbs), on the development of tubes by human endothelial cells (HUVEC).

FIG. 2 is a bar graph depicting an ELISA showing the binding of VEGF-A, IN-02 (e.g., VEGF-DA), and VEGF-D proteins to VEGF-receptors immobilized onto the plate.

FIG. 3 is a graph depicting an ELISA showing the pre- and post-immunization (BL3) sera from three rabbits immunized with IN-02 protein, binding to immobilized immunogen.

FIG. 4 is a graph depicting an ELISA showing the pre- and post-immunization (BL3) sera (caprylic acid purified) from three rabbits immunized with IN-02 protein binding to immobilized rCTB.

FIG. 5 is a graph depicting an ELISA showing the pre- and post-immunization (BL3) sera from three rabbits immunized with IN-02 protein binding to immobilized VEGF-A.

FIG. 6 is a graph depicting an ELISA showing the pre- and post-immunization (BL3) sera from three rabbits immunized with IN-02 protein binding to immobilized VEGF-D.

FIG. 7 is a bar graph depicting the results of a tube formation assay conducted with caprylic acid-purified sera from rabbits immunized with IN-02 protein.

FIG. 8 is a bar graph depicting the results of a HUVEC tube formation assay performed on IN-02 protein that had been stored at 4° C. for one month.

### DETAILED DESCRIPTION OF THE DISCLOSURE

The present disclosure is based, at least in part, on the discovery that chimeric synthetic proteins/molecules including one or more protein domains from a growth factor (e.g., VEGF-A, VEGF-B, VEGF-C, VEGF-D, and the like), one or more linker regions, and one or more immunogenic domains may be used as therapeutic molecules to treat a variety of diseases such as, for example, cancer. The chimeric synthetic molecules provide several unexpected advantages over the prior art. For example, unlike prior art human Epidermal Growth Factor (hEGF) molecules (e.g., U.S. Pat. No. 5,984,018 to Davila et al.) that are present in heterogeneous mixtures containing up to 12 different molecular species, the synthetic proteins/molecules described herein may be produced as a single molecule (e.g., a homogenous population of molecules). Additionally, the synthetic proteins/molecules described herein include ten active components per molecule (although the active components may be increased or decreased in multiples of 5, e.g., as part of a pentamer), whereas prior art hEGF molecules (e.g., U.S. Pat. No. 5,984,018 to Davila et al.) are highly variable in the number of active components present per molecule (e.g., the mean number of active components per molecule of Davila is 1.5). Moreover, the chimeric synthetic proteins/molecules described herein are much more straightforward to manufacture. For example, prior art hEGF molecules (e.g., U.S. Pat. No. 5,984,018) are made by chemically conjugating rP64k and recombinant human EGF (rhEGF) to produce a final molecule that consists of two molecules chemically conjugated to one another. This is in sharp contrast to the synthetic proteins/molecules described herein, which are a single synthetic molecule. Additionally, the chimeric synthetic proteins/molecules described herein have the ability to function as stabilizing scaffolds that better enable human proteins (e.g., growth factors) that are incorporated into the proteins/molecules to adopt native configurations when expressed (e.g., fold properly). Additionally, the chimeric synthetic proteins/molecules described herein have the ability to generate stabilized chimeric synthetic proteins/molecules that have long storage shelf lives. Advantageously, the techniques herein provide novel chimeric synthetic proteins that may be used therapeutically to treat diseases such as, for example, cancer (e.g., cancer vaccines) with a higher immunogenic activity level than prior art methods (e.g., U.S. Pat. No. 5,984,018).

### Overview

Cancer immunology is the study of interactions between an immune system and cancer cells such as, for example, tumors or malignancies. The initiation of an immune response, such as recognition of cancer-specific antigens that are expressed by human tumors and not expressed in normal tissues, is of particular interest. Generally, methods to control the division and proliferation of the malignant cells have focused on isolating these antigens and presenting them so that they are recognized by the immune system as non-self antigens to induce a specific immune response.

There are a significant number of growth factors identified at present, and most, if not all, have been shown to be important mediators of cell proliferation in various cancers in addition to being implicated in other disease conditions. Generally, growth factors are soluble serum proteins that recognize and bind to a group of growth factor receptors located on cell surfaces. Particular growth factors may be specific for a single receptor, or may bind to more than one closely related receptor with varying affinities. Similarly, some receptors bind to only a single growth factor ligand while others can bind to multiple related growth factors, again usually with differing affinities. Upon binding to its natural receptor, the cytoplasmic domain of the receptor is phosphorylated, and this initiates an intra-cellular signaling cascade that results in modulation of transcription of one or more genes and ultimately to progression through the cell cycle and cell proliferation.

Growth factors and their receptors are essential components of the normal processes of growth, development and repair, and their tissue distribution profiles and expression levels closely regulate cell growth. Numerous studies have shown that growth factors can stimulate proliferation of a variety of cell types both in vitro and in vivo (Cohen S., Carpenter G., PNAS USA 72, 1317, 1975, Witsch E et al: Physiology: 25(2):85-101, (2010)). Moreover, certain growth factors have been shown to stimulate proliferation in some cancer cell lines. For example epidermal growth factor (EGF) can stimulate some non-small cell lung carcinoma cells (Osborne C. K. et al. Can Res. 40, 2. 361 (1980)). Other growth factors such as vascular endothelial growth factor (VEGF), fibroblast growth factor (FGF), and platelet-derived growth factor (PDGF) are important in several oncology diseases, such as non-small cell lung cancer (NSCLC) (Ballas M S, Chachoua A., Onco Targets and Therapy: 4, 43-58 (201 1)), prostate cancer, (Cox M E et al; Prostate 69 (0:33-40 (2009)), and breast cancer (Law J et al, Cancer Res; 68,24: 10238-10346 (2008)).

High levels of various growth factor receptors have been reported in malignant tissues. For example, the epidermal growth factor receptor (EGFR) has been detected at unusually high levels in malignant tumors of epithelial origin, such as lung, breast, bladder, ovarian, vulva, colonic, pulmonary, brain and esophagus cancers. The role played by growth factors and their receptors in regulating tumor growth is unknown, but there are suggestions that growth factor receptor expression in tumor cells provides a mechanism for autocrine growth stimulation which leads to uncontrolled proliferation (Schlessinger J., Schreiber A. B., Levi A., Liberman T., Yarden Y. Crit. Rev. Biochem. 1983, 14 (2) 93-1 11). Further, Liao Y et al; Hum Pathol 36(1 1): 1186-1 196 (2005) and Cox M E et al; Prostate: 69(1) 33-40 (2009) describe the role of increased Insular receptor and growth factor on metastatic prostate cancer.

One treatment strategy to target growth factor signaling in cancer therapy has been to use a passive immunotherapy (e.g., monoclonal antibodies) against the particular receptor/receptors involved. Such studies have demonstrated that the specific recognition by an antibody of the receptor that is able to inhibit the binding of the ligand can have an inhibitory effect on the mitogenic stimulation of malignant cells (SATO J. D., et al. Methods in Enzymology, vol. 146 pp 63-81, 1987). However, antibodies that are of murine origin will usually produce a human anti-mouse antibody response (HAMA), thus limiting them to a single administration.

Other treatment strategies have been to use an active immunotherapy with vaccines that contain the growth factor of interest to induce an immune response against the molecule to inhibit the proliferation effect of the growth factor on tumors. U.S. Pat. No. 5,984,018, to Davila et al, entitled Vaccine Composition Comprising Autologous Epidermal Growth Factor or a Fragment or a Derivative Thereof having Anti-tumor Activity and use Thereof in the Therapy of Malignant Diseases, discloses, for example, the use of a vaccine that contains a mixture of a growth factor and an immunogenic (i.e. non-human) carrier protein chemically conjugated together using glutaraldehyde. However, without being bound to any particular theory it is thought that chemical conjugation hinders immune responses against the vaccine.

This is a technically challenging approach, as it requires that the host generates an immune response to a ‘self antigen’, and vertebrate immune systems have evolved to prevent such responses from occurring. Where a strong immune response is generated against a self-antigen, for example, one that includes T-helper cell activation, an auto-immune disease state usually results. For many years it has been hypothesized that some auto-immune disorders, for example, lupus, multiple sclerosis (MS), diabetes etc., might be caused by early exposure to an environmental agent that includes immunogenic epitopes (T-cell epitopes) that closely mimic host self-epitopes. This could lead to the stimulation of T-helper cells that are cross reactive with host epitopes. Subsequent exposure to the environmental agent could then result in an anti-self immune response (Albert, L. J., and Inman, R. D New England Journal of Medicine, December 30th pp 2068-2074, 1999). It has since been demonstrated that a viral antigen can indeed generate an anti-self immune response against a nerve cell protein (Levin, M. C. et. al, Nature Medicine vol 8 (5) pp 509-513, 2002).

U.S. Publ. No. 2006/0251654, to Casimiro et al, entitled Method for Treatment of Malignant and Infectious Chronic Diseases, (the '654 publication) discloses a method of treating a subject bearing a malignant or infectious chronic disease comprising the method of immunizing the subject with a vaccine containing a self-antigen associated with the malignant or infectious chronic disease that is coupled to a carrier protein; treating the subject with an immune modulator agent; and immunizing the subject again with the vaccine of the step 1, and an appropriate adjuvant selected from aluminum hydroxide and Montanide ISA 51 (Seppic, Paris, France). Unfortunately, the preparation of the vaccine by chemical conjugation is thought to hinder the immune response.

The majority of the vaccines described above exhibit a number of limitations, arising primarily from the method of manufacture and the potential lack of uniformity and homology of the protein product. The vaccines described above generally comprise a mixture of a recombinant carrier protein and polypeptides of human origin that are chemically conjugated using glutaraldehyde. Unfortunately, this reactive reagent can undesirably form covalent cross-linking bonds between varieties of chemical groups, and generally leads to a highly heterogeneous product. Thus, the resulting vaccines may comprise not only carrier protein molecules with varying numbers of the target human polypeptide attached (for example, 0, 1, 2, 3 etc.), but the human polypeptides can each be attached to the carrier via different atoms and so in different positions and in different orientations. Furthermore, both the target polypeptide and carrier protein molecules may be conjugated to themselves, resulting in various homo-multimers that may have no clinical efficacy and may not contribute to an anti-cancer patient immune response.

### Synthetic Proteins/Molecules

The present disclosure provides a homogeneous synthetic protein/molecule for improving the presentation of the maximum number of growth factor epitopes, tumor antigen epitopes, and/or receptor binding sites as elements of an immunogenic synthetic protein/molecule. In one illustrative embodiment, a synthetic protein/molecule expressing all or portions of an immunogenic carrier domain (e.g., cholera toxin B (CT-B)), and a synthetic epidermal growth factor (sEGF), a tumor antigen, and/or a receptor is described. In alternative illustrative embodiments, the protein may express other immunogenic synthetic or recombinant proteins/molecules that are modeled based upon known immunogenic proteins. It is contemplated within the scope of the disclosure that such synthetic proteins/molecules may express polypeptides that are highly immunogenic to the human immune system. Preferably, the synthetic proteins/molecules confer additional properties to the chimeric protein such as, for example, high expression yield and ease of manufacture, oral stability and the ability to cross from gut to blood stream, and/or previous safe use in humans.

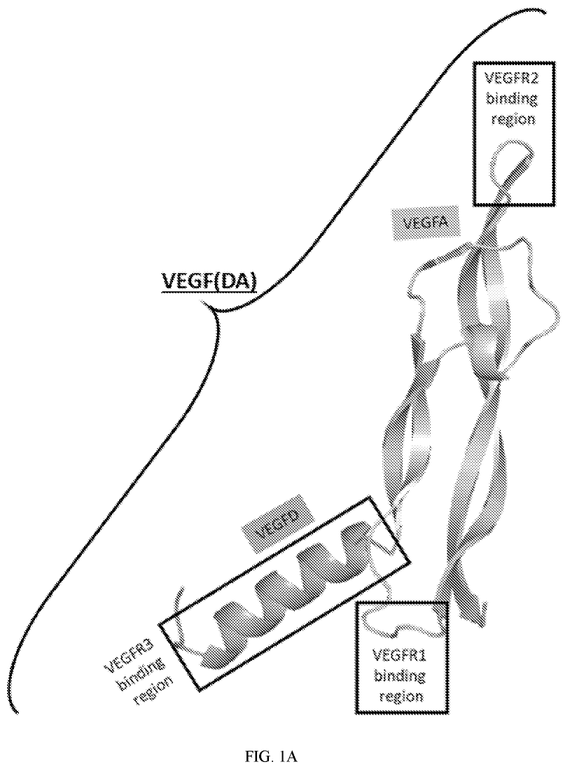

In an illustrative embodiment, the synthetic proteins/molecules disclosed herein may include or express a high proportion of a protein sequence derived from target self antigens, as a function of total molecular weight. This may be achieved, for example, by using a large protein model containing multiple growth factor epitopes. These growth factor epitopes may be multiple copies of whole or part of a single growth factor, or copies of whole or part of more than one different growth factor. These growth factor epitopes may be naturally occurring or synthetic (e.g., artificial). For example, BVN22E (also referred to as IN01), an illustrative synthetic protein described herein, may have a molecular weight of about 120 kD. In an illustrative embodiment, the growth factor epitopes described herein may correspond to one or more domains within the growth factor (e.g., EGF targeted signaling pathway (TSP) domains). In an illustrative embodiment, an EGF domain may include the region which presents or constrains the n-loop, e.g., the region defined by about cysteine 6 to about cysteine 42, the region defined by about cysteine 6 to about cysteine 31 or the region defined by about cysteine 22 about cysteine 33 or the region defined by about cysteine 22 about cysteine 31 or the region defined by about cysteine 62 about cysteine 14 of the synthetic protein sequence (e.g., FIG. 1A). Without being bound by any particular theory, it is contemplated within the scope of the disclosure that different regions or sub-regions between cysteine 6 and cysteine 42 may have beneficial effects when incorporated into the synthetic proteins/molecules of the disclosure. For example, the following regions may have beneficial effects: the region between cysteine 6 and cysteine 14, the region between cysteine 6 and cysteine 20, the region between cysteine 6 and cysteine 31, the region between cysteine 6 and cysteine 33, and the region between cysteine 6 and cysteine 42. It is also contemplated within scope of the disclosure that the reverse progressive sequence may also be beneficial. For example, the following regions may have beneficial effects: the region between cysteine 42 and cysteine 33, the region between cysteine 42 and cysteine 31, the region between cysteine 42 and cysteine 20, the region between cysteine 42 and cysteine 14, and the region between cysteine 42 and cysteine 6. It is further contemplated within the scope of the invention that specific intervals within the region between cysteine 6 and cysteine 42 may provide beneficial effects when incorporated into the synthetic proteins/molecules of the disclosure (e.g., the region between cysteine 6 and cysteine 14, the region between cysteine 14 and cysteine 20, the region between cysteine 20 and cysteine 31, and the region between cysteine 33 and cysteine 42).

According to the disclosure, the expressions of the growth factor epitopes should be folded allowing their natural conformation to be substantially retained and presented to components of the host immune system in such a way as to elicit a robust host immune response to said epitopes. Examples of suitable natural protein models to model an epitope supporting domain of a synthetic proteins/molecules include, but are not limited to, cholera toxin B sub-unit, E. coli heat-labile LT and LT-II enterotoxin B subunits, veratoxin, pertussis toxin, C. jejuni enterotoxin, Shiga toxin, listeria toxin, tetanus toxoid, diphtheria toxoid, N. meningitidisl outer membrane protein, bacteriophage coat protein, adenovirus and other viral coat proteins. Alternatively, a non-self component of the protein can be small. At a minimum, the non-self sequence(s) should comprise about 9, 10, 11 or more amino acids in length, and include either entirely or in-part at least one human T-cell epitope. As described herein, non-natural synthetic polypeptides (e.g., BVN22E, IN01) may be used that fulfill the requirements of conferring immunogenicity to the whole protein and allowing appropriate presentation of growth factors, receptors, tumor antigens or epitopes thereof to the host immune system.

According to the disclosure, the synthetic proteins/molecules provided herein—whether growth factors or parts thereof, cellular receptors or parts thereof, or tumor antigens or parts thereof—are related to a broad range of cellular pathways involved in chronic disease, growth factor based or receptor based cancers, and/or solid tumors for use as tumor antigens within the said synthetic proteins. The proteins are in the form of a synthetic proteins/molecules and may be useful in treating chronic diseases, for example, breast, lung, bladder, ovarian, vulva, colonic, pulmonary, brain, colorectal, intestinal, head and neck, and esophagus cancers. As different tumor antigens can be expressed and multiple cellular receptors and growth factors over expressed in the said diseases, the proteins described hereunder can contain one or more different tumor antigens, one or more different receptors or growth factors of one or multiple cellular pathways associated with the disease. These proteins are called multivalent.

In an illustrative embodiment, a protein comprised of a homogeneous synthetic proteins/molecules expressing one or more epidermal growth factor (EGF) neutralizing domains (e.g., TSP domains) is disclosed. The protein may be in the form of a synthetic proteins/molecules and may be useful in treating chronic diseases, for example, breast, lung, bladder, ovarian, vulva, colonic, pulmonary, brain, colorectal, head and neck, and esophagus cancers. In an illustrative embodiment, the protein is a synthetic proteins/molecules expressing or including synthetic EGF sequences and CT-B sequences, as shown in FIG. 1A. In an illustrative embodiment, a growth factor component of the synthetic protein sequence may include a sequence that is less than 80% identical to EGF. For example, a growth factor component may include an EGF sequence with 11 amino acid substitutions that may increase the immunogenicity of the growth factor portion of the synthetic protein sequence. Without being bound by theory, it is believed that the region of EGF that ‘presents’ or constrains the β-loop (e.g., the region defined by Cys6 to Cys31) may be an important to include in the synthetic protein and amenable to target for amino acid modification. In an illustrative embodiment, regions outside of Cys6 to Cys31 may also be targeted for modification (e.g., E11 and A12).

In an illustrative embodiment, the TSP1 and TSP2 domains of hEGF may be modified as shown in FIG. 1B to create synthetic EGF (sEGF) regions to be included in a synthetic protein/molecule herein.

In an illustrative embodiment, the synthetic proteins/molecules disclosed herein may include all, or a portion of, growth factors including without limitation grow factors such as, for example, Neuregulin (NRG1β), Transforming Growth Factor α (TGFα), Vascular endothelial growth factor (VEGF), and the like.

In other illustrative embodiments, the synthetic proteins/molecules described herein may include one or more linkers or spacers. One or more of the embodiments described above include sEGF fused to CT-B such that the sEGF portion of the synthetic molecule is separated from the CT-B portion by a GGSGGTSGGGGGSG linker. These resulting recombinant or chimeric proteins essentially included sEGF fused directly to CT-B. In other illustrative embodiments, the EGF and CT-B components of the chimeric protein are effectively separated by 3 to 14 amino acids, which form a flexible spacer or linker between the two domains. It is contemplated within the scope of the disclosure that the linker may be 3, 4, 5, 6, 7, 8, 9, 10, 11, 12, 13, or 14 amino acids in length. In some cases in which a growth factor has a larger size (e.g., human growth factor), it may be useful to use a longer linker sequence. The following exemplary linkers may be used and include, but are not limited to, the following: SSG, SSGGG, SGG, GSSG, GGSGG, GGGGS, SSGGGSGG, SSGGGGSGGG, TSGGGSG, TSGGGGSGG, SSGGGSGGSSG, GGSGGTSGGGSG, SGGTSGGGGSGG, GGSGGTSGGGGSGG, SSGGGGSGGGSSG, SSGGGSGGSSGGG, and SSGGGGSGGGSSGGG. One of skill in the art will appreciate that there are many other sequences/combinations of primarily ‘G’ and ‘S’ that would also serve as useful linker sequences.

Without being bound by any particular theory, it is contemplated that the synthetic proteins/molecules disclosed herein provide significant clinical benefits. For example, the synthetic proteins/molecules disclosed herein may be expressed in bacterial systems at commercial scale and purity, while producing stable polypeptides that fold correctly and are functional. Additionally, the synthetic proteins/molecules disclosed herein are able to form a pentamers. Additionally, the synthetic proteins/molecules disclosed herein have the advantageous property of requiring much lower levels of protein for vaccination because the amount of carrier necessary significantly lower than prior art methods (e.g., U.S. Pat. No. 5,984,018 to Davila et al.). In this regard, the synthetic proteins/molecules disclosed herein are able to deliver more growth factor to a patient in a significantly lower volume of vaccine.

### Adjuvant

Certain illustrative embodiments as provided herein include synthetic proteins/molecules according to the disclosure within vaccine compositions and immunological adjuvant compositions, including pharmaceutical compositions, that contain, in addition to synthetic proteins/molecules at least one adjuvant, which refers to a component of such compositions that has adjuvant activity. An adjuvant having such adjuvant activity includes a composition that, when administered to a subject such as a human (e.g., a human patient), a non-human primate, a mammal or another higher eukaryotic organism having a recognized immune system, is capable of altering (i.e., increasing or decreasing in a statistically significant manner, and in certain preferred embodiments, enhancing or increasing) the potency and/or longevity of an immune response. In certain illustrative embodiments disclosed herein a desired antigen and or antigens contain within a protein carrier, and optionally one or more adjuvants, may so alter, e.g., elicit or enhance, an immune response that is directed against the desired antigen and or antigens which may be administered at the same time or may be separated in time and/or space (e.g., at a different anatomic site) in its administration, but certain illustrative embodiments are not intended to be so limited and thus also contemplate administration of synthetic proteins/molecules in a composition that does not include a specified antigen but which may also include but is not limited to one or more co-adjuvant, an imidazoquinline immune response modifier.

Accordingly and as noted above, adjuvants include compositions that have adjuvant effects, such as saponins and saponin mimetics, including QS21 and QS21 mimetics (see, e.g., U.S. Pat. No. 5,057,540; EP 0 362 279 B1; WO 95/17210), alum, plant alkaloids such as tomatine, detergents such as (but not limited to) saponin, polysorbate 80, Span 85 and stearyl tyrosine, one or more cytokines (e.g., GM-CSF, IL-2, IL-7, IL-12, TNF-alpha, IFN-gamma), an imidazoquinoline immune response modifier, and a double stem loop immune modifier (dSLIM, e.g., Weeratna et al, 2005 Vaccine 23:5263).

Detergents including saponins are taught in, e.g., U.S. Pat. No. 6,544,518; Lacaille-Dubois, M and Wagner H. (1996 Phytomedicine 2:363-386), U.S. Pat. No. 5,057,540, Kensil, Crit. Rev Ther Drug Carrier Syst, 1996, 12 (1-2): 1-55, and EP 0 362 279 B1. Particulate structures, termed Immune Stimulating Complexes (ISCOMS), comprising fractions of Quil A (saponin) are hemolytic and have been used in the manufacture of vaccines (Morein, B., EP 0 109 942 B 1). These structures have been reported to have adjuvant activity (EP 0 109 942 B 1; WO 96/1 1711). The hemolytic saponins QS21 and QS17 (HPLC purified fractions of Quil A) have been described as potent systemic adjuvants, and the method of their production is disclosed in U.S. Pat. No. 5,057,540 and EP 0 362 279 B1. Also described in these references is the use of QS7 (a non-hemolytic fraction of Quil-A) which acts as a potent adjuvant for systemic vaccines. Use of QS21 is further described in Kensil et al. (1991. J. Immunology 146:431-437). Combinations of QS21 and polysorbate or cyclodextrin are also known (WO 99/10008). Particulate adjuvant systems comprising fractions of QuilA, such as QS21 and QS7 are described in WO 96/33739 and WO 96/1 1711. Other saponins which have been used in systemic vaccination studies include those derived from other plant species such as Gypsophila and Saponaria (Bomford et al, Vaccine, 10(9):572-577, 1992). Escin is another detergent related to the saponins for use in the adjuvant compositions of the embodiments herein disclosed. Escin is described in the Merck index (12.sup.th Ed. entry 3737) as a mixture of saponin occurring in the seed of the horse chestnut tree, Aesculus hippocastanum. Its isolation is described by chromatography and purification (Fiedler, Arzneimittel-Forsch. 4, 213 (1953)), and by ion-exchange resins (Erbring et al, U.S. Pat. No. 3,238,190). Fractions of escin (also known as aescin) have been purified and shown to be biologically active (Yoshikawa M, et al. (Chem Pharm Bull (Tokyo) 1996 August; 44(8): 1454-1464)). Digitonin is another detergent, also being described in the Merck index (12th Ed., entry 3204) as a saponin, being derived from the seeds of Digitalis purpurea and purified according to the procedure described by Gisvold et al, J. Am. Pharm. Assoc., 1934, 23, 664; and Rubenstroth-Bauer, Physiol. Chem., 1955, 301, 621.

Other adjuvants or co-adjuvants for use according to certain herein disclosed embodiments include a block co-polymer or biodegradable polymer, which refers to a class of polymeric compounds with which those in the relevant art will be familiar. Examples of a block co-polymer or biodegradable polymer that may be included in a vaccine composition or an immunological adjuvant include Pluronic® L121 (BASF Corp., Mount Olive, N.J.; see, e.g., Yeh et al, 1996 Pharm. Res. 13: 1693).

Certain further illustrative embodiments contemplate immunological adjuvants that include but are not limited to an oil, which in some such embodiments may contribute co-adjuvant activity and in other such embodiments may additionally or alternatively provide a pharmaceutically acceptable carrier or excipient. Any number of suitable oils are known and may be selected for inclusion in vaccine compositions and immunological adjuvant compositions based on the present disclosure. Examples of such oils, by way of illustration and not limitation, include squalene, squalane, mineral oil, olive oil, cholesterol, and a mannide monooleate.

Immune response modifiers such as imidazoquinoline immune response modifiers are also known in the art and may also be included as adjuvants or co-adjuvants in certain presently disclosed embodiments.

As also noted above, one type of adjuvant or co-adjuvant for use in a vaccine composition according to the disclosure as described herein may be the aluminum co-adjuvants, which are generally referred to as “alum.” Alum co-adjuvants are based on the following: aluminum oxy-hydroxide; aluminum hydroxyphosphoate; or various proprietary salts. Alum co-adjuvants are be advantageous because they have a good safety record, augment antibody responses, stabilize antigens, and are relatively simple for large-scale production. (Edelman 2002 Mol. Biotechnol. 21: 129-148; Edelman, R. 1980 Rev. Infect. Dis. 2:370-383.)

### Pharmaceutical Compositions

In certain illustrative embodiments, the pharmaceutical composition is a vaccine composition that comprises both the synthetic proteins/molecules according to the disclosure and may further comprise one or more components, as provided herein, that are selected from TLR agonist, co-adjuvant (including, e.g., a cytokine, an imidazoquinoline immune response modifier and/or a dSLIM) and the like and/or a recombinant expression construct, in combination with a pharmaceutically acceptable carrier, excipient or diluent.

Illustrative carriers will be nontoxic to recipients at the dosages and concentrations employed. For vaccines comprising synthetic proteins/molecules, about 0.01 μg/kg to about 100 mg/kg body weight will be administered, typically by the intradermal, subcutaneous, intramuscular or intravenous route, or by other routes.

It will be evident to those skilled in the art that the number and frequency of administration will be dependent upon the response of the host. “Pharmaceutically acceptable carriers” for therapeutic use are well known in the pharmaceutical art, and are described, for example, in Remington's Pharmaceutical Sciences, Mack Publishing Co. (A. R. Gennaro edit. 1985). For example, sterile saline and phosphate-buffered saline at physiological pH may be used. Preservatives, stabilizers, dyes and even flavoring agents may be provided in the pharmaceutical composition. For example, sodium benzoate, ascorbic acid and esters of p-hydroxybenzoic acid may be added as preservatives. In addition, antioxidants and suspending agents may be used.

The pharmaceutical compositions may be in any form which allows for the composition to be administered to a patient. For example, the composition may be in the form of a solid, liquid or gas (aerosol). Typical routes of administration include, without limitation, oral, topical, parenteral (e.g., sublingually or buccally), sublingual, rectal, vaginal, and intranasal (e.g., as a spray). The term parenteral as used herein includes iontophoretic sonophoretic, passive transdermal, microneedle administration and also subcutaneous injections, intravenous, intramuscular, intrasternal, intracavernous, intrathecal, intrameatal, intraurethral injection or infusion techniques. In a particular embodiment, a composition as described herein (including vaccine and pharmaceutical compositions) is administered intradermally by a technique selected from iontophoresis, microcavitation, sonophoresis or microneedles.

The pharmaceutical composition is formulated so as to allow the active ingredients contained therein to be bioavailable upon administration of the composition to a patient. Compositions that will be administered to a patient take the form of one or more dosage units, where for example, a tablet may be a single dosage unit, and a container of one or more compounds of the invention in aerosol form may hold a plurality of dosage units.

For oral administration, an excipient and/or binder may be present. Examples are sucrose, kaolin, glycerin, starch dextrins, sodium alginate, carboxymethylcellulose and ethyl cellulose. Coloring and/or flavoring agents may be present. A coating shell may be employed.

The composition may be in the form of a liquid, e.g., an elixir, syrup, solution, emulsion or suspension. The liquid may be for oral administration or for delivery by injection, as two examples. When intended for oral administration, preferred compositions contain one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

A liquid pharmaceutical composition as used herein, whether in the form of a solution, suspension or other like form, may include one or more of the following carriers or excipients: sterile diluents such as water for injection, saline solution, preferably physiological saline, Ringer's solution, isotonic sodium chloride, fixed oils such as squalene, squalane, mineral oil, a mannide monooleate, cholesterol, and/or synthetic mono or digylcerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. An injectable pharmaceutical composition is preferably sterile.

In a particular embodiment, a pharmaceutical or vaccine composition of the invention comprises a stable aqueous suspension of less than 0.2 um and further comprises at least one component selected from the group consisting of phospholipids, fatty acids, surfactants, detergents, saponins, fluorodated lipids, and the like.

It may also be desirable to include other components in a vaccine or pharmaceutical composition, such as delivery vehicles including but not limited to aluminum salts, water-in-oil emulsions, biodegradable oil vehicles, oil-in-water emulsions, biodegradable microcapsules, and liposomes. Examples of additional immunostimulatory substances (co-adjuvants) for use in such vehicles are also described above and may include N-acetylmuramyl-L-alanine-D-isoglutamine (MDP), glucan, IL-12, GM-CSF, gamma interferon and IL-12.

While any suitable carrier known to those of ordinary skill in the art may be employed in the pharmaceutical compositions of this invention, the type of carrier will vary depending on the mode of administration and whether a sustained release is desired. For parenteral administration, such as subcutaneous injection, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g., polylactic galactide) may also be employed as carriers for the pharmaceutical compositions of this invention.

Pharmaceutical compositions may also contain diluents such as buffers, antioxidants such as ascorbic acid, low molecular weight (less than about 10 residues) polypeptides, proteins, amino acids, carbohydrates including glucose, sucrose or dextrins, chelating agents such as EDTA, glutathione and other stabilizers and excipients. Neutral buffered saline or saline mixed with nonspecific serum albumin are exemplary appropriate diluents. Preferably, product may be formulated as a lyophilizate using appropriate excipient solutions (e.g., sucrose) as diluents.

In an illustrative embodiment, the epitope or receptor supporting domain of the synthetic protein/molecule, whether derived from a natural or synthetic polypeptide sequence, should have the capacity to self-assemble into oligomeric multimers under appropriate chemical/environmental conditions, or to be reduced to monomers under alternative conditions. Ideally, multimerisation domains will assemble into stable multimers with a discreet number of sub-units, for example dimers, trimers, tetramers, pentamers, etc., such that a product of homogeneous size is generated. Examples of natural polypeptides include, but are not limited to, leucine zippers, lac repressor protein, streptavidin/avidin, cholera toxin B sub-unit, Pseudomonas trimerization domain, and viral capsid proteins.

In an illustrative embodiment, a process of preparing a multivalent molecule is disclosed. In this illustrative embodiment, the process includes assembling multimers from monomeric sub-units to form a synthetic protein including one or more tumor antigens, receptors, and/or a growth factors or parts thereof.

In another illustrative embodiment, a process of preparing a vaccine formulation is disclosed. In this illustrative embodiment, the process includes mixing one or more single monovalent multimers together preparing a multivalent vaccine including a synthetic protein/molecule including one or more tumor antigens, receptors, and/or a growth factors or parts thereof.

In yet another illustrative embodiment, a process for treating a patient is disclosed. In this illustrative embodiment, the process includes administering separately to the patient one or more monovalent, one tumor antigen, receptor, and/or growth factor, recombinant proteins in a same day or at alternate days or times during a vaccination period.

While the synthetic protein/molecule is described as including or expressing one or more of all or a portion of at least one sequence of the tumor antigens, the growth factors, and/or the receptors, and the CT-B sequence, the synthetic protein/molecule may include the natural CT-B sequence or a sequence substantially similar to the natural CT-B sequence and/or a synthetic sequence. While the synthetic protein/molecule is described as including or expressing the CT-B sequence, the synthetic protein/molecule may include or express a derivation of the CT-B sequence or a sequence that is substantially similar to the CT-B sequence.

While the homogeneous synthetic proteins/molecules expressing or incorporating one or more tumor antigens, synthetic growth factors, and/or receptors have been described and illustrated in connection with certain embodiments, many variations and modifications will be evident to those skilled in the art and may be made without departing from the spirit and scope of the disclosure. The disclosure is thus not to be limited to the precise details of methodology or construction set forth above as such variations and modification are intended to be included within the scope of the disclosure.

### EXAMPLES

The present disclosure is further illustrated by the following examples, which should not be construed as limiting. The contents of all references, GenBank Accession and Gene numbers, and published patents and patent applications cited throughout the application are hereby incorporated by reference. Those skilled in the art will recognize that the disclosure may be practiced with variations on the disclosed structures, materials, compositions and methods, and such variations are regarded as within the scope of the disclosure.

### Example 1: Bi-Specific Chimeric Antigens

A problem that arises when human proteins (e.g., growth factors) or parts thereof are combined with immunogenic carrier molecules such as, for example, cholera toxin B sub-unit (CTB) to create recombinant proteins is that the human protein (e.g., a human growth factor) does not always fold into the correct native configuration. The ability of human proteins within a recombinant protein to fold correctly may vary significantly from protein to protein, even among closely related molecules. For example, epidermal growth factor (EGF) can readily be correctly folded from insoluble inclusion bodies, and is very stable thereafter; however, both transforming growth factor alpha (TGFα) and the EGF-like domain of neuregulin are more difficult to produce in a properly folded form, and are also noticeably less stable.

Vascular endothelial growth factor (VEGF) comprises four structurally related proteins, VEGF-A, VEGF-B, VEGF-C and VEGF-D, that mediate signaling through three receptors, VEGFR-1, VEGFR-2 and VEGFR-3. VEGF-A and VEGF-D signal through VEGFR-1, whereas VEGF-A, VEGF-B and VEGF-C can bind to VEGFR-2. VEGF-C and VEGF-D bind to VEGFR-3, thus showing both similarities and differences in their receptor binding characteristics. All of the VEGF growth factors share a structurally common ‘homology domain,’ which is associated with recognition and binding of VEGFR-1 and VEGFR-2 (VEGF-A, VEGF-B and VEGF-C) and comprises the sequence downstream of the first cysteine residue. The N-terminus of VEGF-A and VEGF-B, upstream of the first cysteine residue, is not involved directly in receptor binding. In contrast, the N-terminus of VEGF-C and VEGF-D is involved in binding to VEGFR-3.

VEGF-A can be expressed in E. coli as insoluble inclusion bodies, and subsequently denatured, solubilized, and refolded into a fully functional protein. VEGF-D is similarly receptive to refolding from inclusion bodies, however, it is much less stable and shows visible signs of degradation after just one week at 4° C., which renders it unsuitable for use in therapeutic applications in its native form. VEGF-C is very difficult to fold correctly either from inclusion bodies, or when expressed as soluble protein in bacteria.

The N-terminus region of VEGF-C and VEGF-D (the sequence upstream of the first cysteine residue) forms an alpha helix in the native protein, which interacts with VEGFR-3. This structure also requires interaction with other parts of the VEGF molecule to adopt and maintain this configuration. When expressed in isolation, or as a genetic fusion with an ‘irrelevant’ carrier, the resulting protein does not exhibit any binding to VEGFR-3. However, when the N-terminal domain of VEGF-A is replaced with the N-terminus of VEGF-D as shown in FIG. 1A, the resulting protein can bind to all three VEGF-receptors and modulate three separate signaling pathways. The VEGF-A domain therefore serves as a stabilizing scaffold, enabling the N-terminal domain of VEGF-D to adopt and maintain its natural structure. The stabilized bi-specific chimeric VEGF, including sequences derived from both VEGF-D and VEGF-A was further fused to the C-terminus of CTB, separated by a 10 amino acid glycine/serine-rich flexible linker. This molecule was designated IN-02 and is shown schematically in FIG. 1B. The protein sequence of IN-02 is shown in FIG. 1C.

In order to analyze the functional characteristics of the VEGF-based molecules, two assays were employed: a Tube Formation Assay (TFA) and an ELISA. The TFA involved culturing Human Umbilical Vein Endothelial Cells (HUVEC) and observing the development of ‘tubes’, representing the formation of blood capillaries, over time. The formation of tubes by cells cultured with stimulatory (growth factors) and inhibitory (neutralizing antibodies) modulators was then compared with those not treated. For the ELISA, recombinant VEGFR (the extracellular domains of VEGF-receptors fused to human IgG Fc region) was coated onto ELISA plates. The plates were incubated with VEGF proteins, and bound VEGF was detected with protein-specific antibodies.

FIG. 1D shows the effect of growth factors and neutralizing antibodies (Nabs) on the development of tubes (vascularisation) by human endothelial cells (HUVEC). Both VEGF-A and VEGF-D were able to stimulate tube formation independently (FIG. 1D, white and gray bars), and this stimulation was prevented by the addition of neutralizing antibodies to each growth factor. The chimeric VEGF-DA protein is also able to stimulate tube formation (first hatched bar). This stimulation can be partially inhibited by neutralizing antibodies to VEGF-A and VEGF-D separately, and more completely when both antibodies are added.

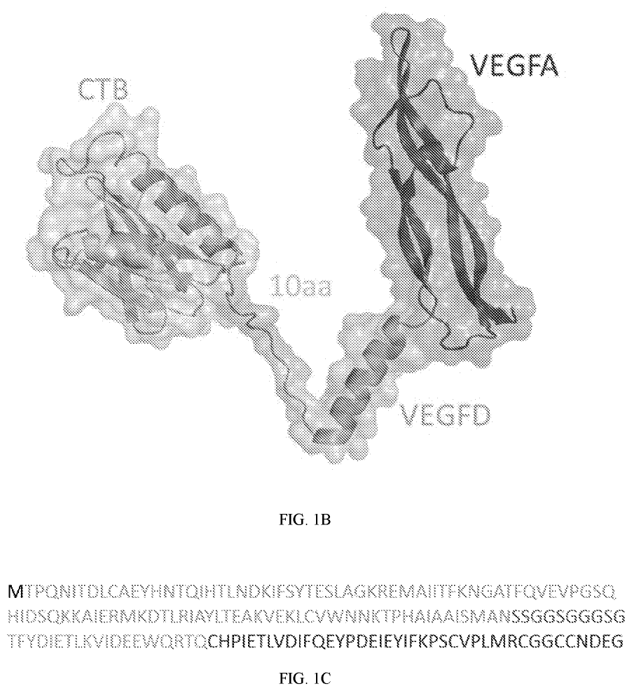

FIG. 2 depicts ELISA data showing the binding of VEGF proteins to VEGF-receptors immobilized onto the plate. Recombinant human VEGF-A bound to receptor-1 and to receptor 2 (bars on left). VEGF-D bound to receptor 3 and to receptor 2 (bars on right). The chimeric IN-02 protein bound to receptors 1, 2 and 3. Binding of IN-02 to receptor 2 could only be detected using an anti-VEGF-A antibody (grey bar), as both receptor-2 and the anti-VEGF-D antibody bound to the same region of the chimeric protein. Three rabbits were immunized with 100 μg IN-02 (subcut.) on days 0, 14, 28 and 56. Bleeds were taken on days 0 (pre-immune), 2, 3, and 56. For clarity, only pre- and bleed 3 data are shown.

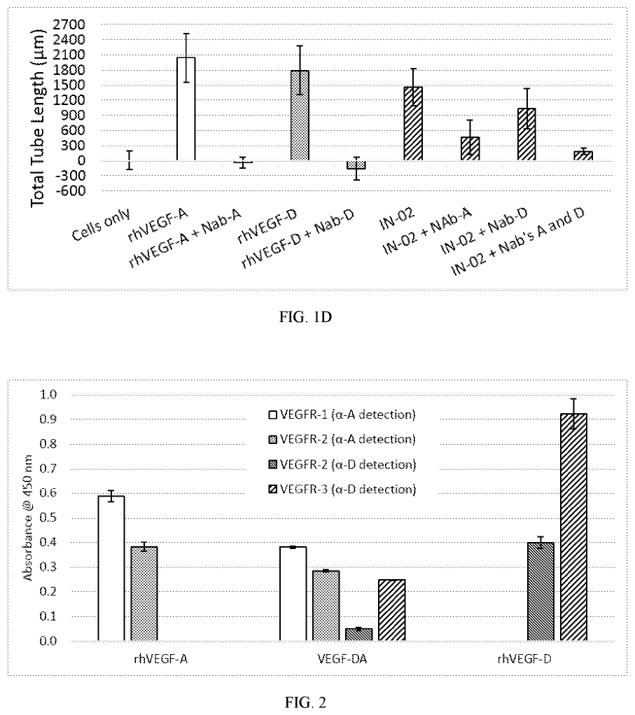

FIG. 3 depicts ELISA data showing the pre- and post-immunization (BL3) sera from three rabbits immunized with IN-02 protein bound to immobilized immunogen that clearly indicated that all three rabbits mounted an immune response to the immunogen and that there was no pre-immunization reactivity.

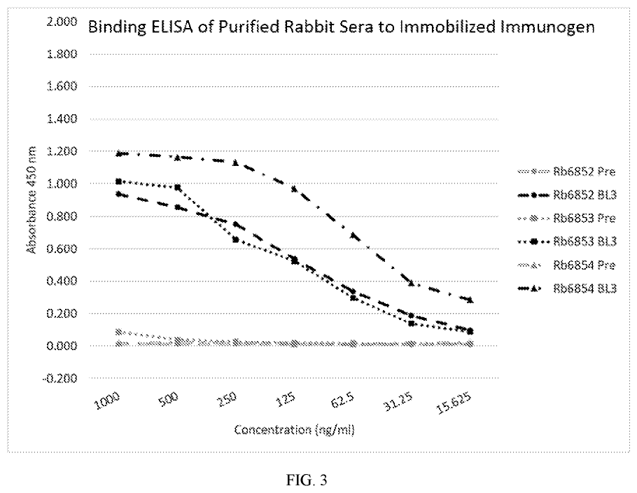

FIG. 4 depicts ELISA data showing the pre- and post-immunization (BL3) sera (caprylic acid purified) from three rabbits immunized with IN-02 protein that bound to immobilized rCTB. All three rabbits mounted an immune response to the CTB domain of the immunogen and that reacted with rCTB. There was no pre-immunization reactivity.

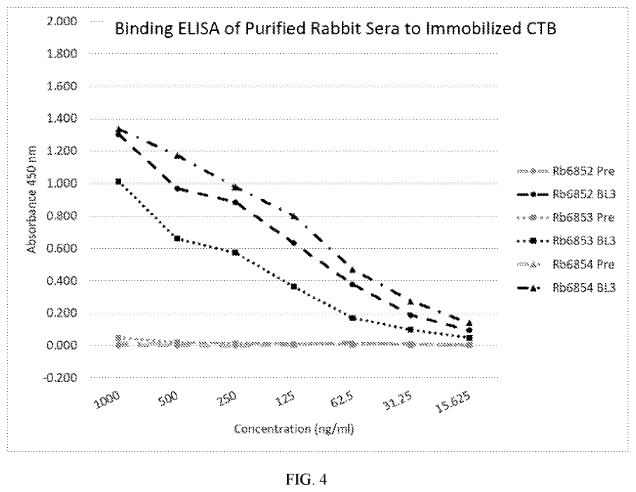

FIG. 5 depicts ELISA data showing the pre- and post-immunization (BL3) sera from three rabbits immunized with IN-02 protein that bound to immobilized VEGF-A. All three rabbits mounted an immune response to the VEGF-A domain of the immunogen and that also reacted with rhVEGF-A. There was no pre-immunization reactivity.

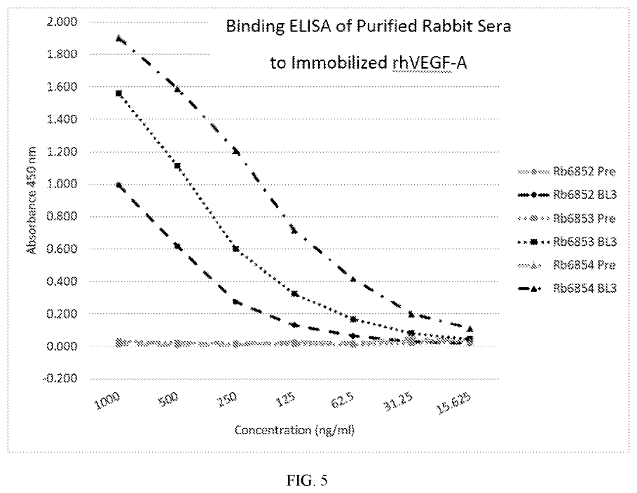

FIG. 6 depicts ELISA showing the pre- and post-immunization (BL3) sera from three rabbits immunized with IN-02 protein that bound to immobilized VEGF-D. All three rabbits mounted an immune response to the VEGF-D domain of the immunogen and that also reacted with rhVEGF-D. There was no pre-immunization reactivity. Following immunization of rabbits with IN-02 protein, all animals generated an immune response against the immunizing antigen, and that response what able to recognize full length native rhVEGF-A and rhVEGF-D in addition to the immunogenic CTB ‘carrier’ domain.

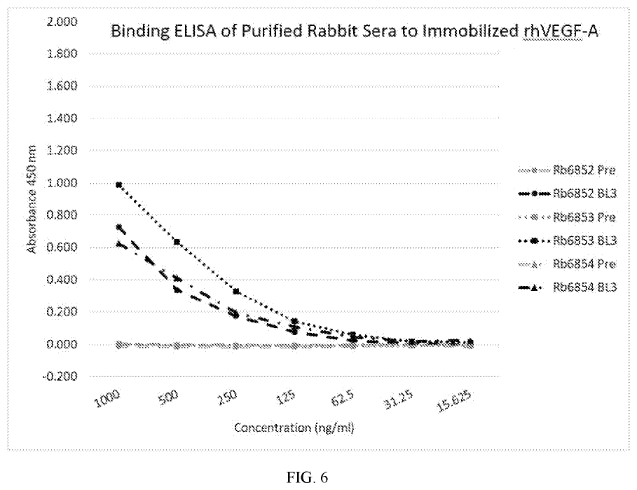

To determine the effectiveness of the immune responses in neutralizing signaling induced by VEGF-A and VEGF-D, a HUVEC tube formation assay was performed as described earlier. FIG. 7 shows that results of a tube formation assay conducted with caprylic acid-purified sera from rabbits immunized with IN-02 protein. All three sera significantly inhibited tube formation induced by co-stimulation with both VEGF-A and VEGF-D simultaneously (black bars).

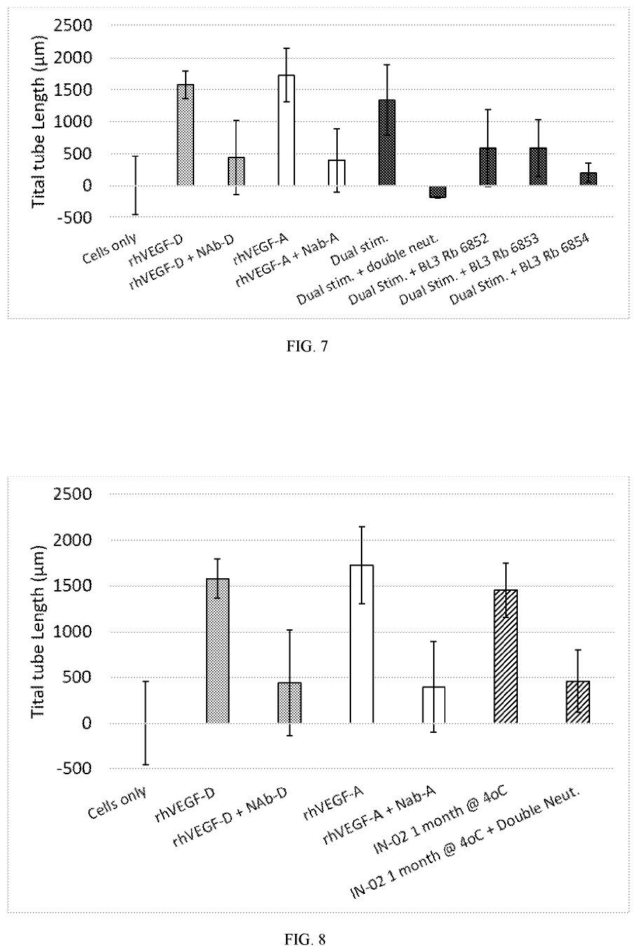

FIG. 8 shows the results of an HUVEC tube formation assay performed on IN-02 protein that had been stored at 4° C. for one month. Despite storage, the IN-02 protein still stimulated tube formation to a similar extent as either VEGF-A or VEGF-D, and that stimulation could be effectively inhibited by simultaneous incubation with antibodies able to neutralize VEGF-A and VEGF-D.

### INCORPORATION BY REFERENCE

All documents cited or referenced herein and all documents cited or referenced in the herein cited documents, together with any manufacturer's instructions, descriptions, product specifications, and product sheets for any products mentioned herein or in any document incorporated by reference herein, are hereby incorporated by reference, and may be employed in the practice of the disclosure.

### EQUIVALENTS

It is understood that the detailed examples and embodiments described herein are given by way of example for illustrative purposes only, and are in no way considered to be limiting to the disclosure. Various modifications or changes in light thereof will be suggested to persons skilled in the art and are included within the spirit and purview of this application and are considered within the scope of the appended claims. Additional advantageous features and functionalities associated with the systems, methods, and processes of the present disclosure will be apparent from the appended claims. Moreover, those skilled in the art will recognize, or be able to ascertain using no more than routine experimentation, many equivalents to the specific embodiments of the disclosure described herein. Such equivalents are intended to be encompassed by the following claims.

We claim:

1. A chimeric synthetic protein, comprising:
a chimeric polypeptide sequence;
at least one linker, and
a polypeptide sequence.

2. The chimeric synthetic protein according to claim 1, wherein the polypeptide sequence includes an immunogenic polypeptide sequence.

3. The chimeric synthetic protein according to claim 1, wherein the polypeptide sequence includes a cholera toxin B (CT-B) protein.

4. The chimeric synthetic protein according to claim 1, wherein the at least one linker includes a first linker that separates the chimeric polypeptide sequence from the polypeptide sequence.

5. The chimeric synthetic protein according to claim 4, wherein the first linker is selected from the group consisting of SSG, GSSG, SSGGG, SGG, GGSGG, GGGGS, SSGGGSGG, SSGGGGSGGG, TSGGGSG, TSGGGGSGG, SSGGSGGGSG, SSGGGSGGS SG, GGSGGTSGGGSG, SGGTSGGGGSGG, GGSGGTSGGGGSGG, SSGGGGSGGGSSG, SSGGGSGGSSGGG, and SSGGGGSGGGSSGGG.

6. The chimeric synthetic protein according to claim 4, wherein the first linker is SSGGSGGGSG.

7. The chimeric synthetic protein according to claim 1, wherein the chimeric polypeptide sequence includes a vascular endothelial growth factor (VEGF) sequence.

8. The chimeric synthetic protein according to claim 1, wherein the chimeric polypeptide sequence includes a VEGF sequence selected from the group consisting of VEGF-A, VEGF-B, VEGF-C, VEGF-D, and combinations thereof.

9. The chimeric synthetic protein according to claim 8, wherein the chimeric polypeptide sequence includes a first VEGF domain and a second VEGF domain.

10. The chimeric synthetic protein according to claim 9, wherein the first VEGF domain includes VEGF-D, or a portion thereof, and the second VEGF domain includes VEGF-A, or a portion thereof.

11. The chimeric synthetic protein according to claim 10, wherein the first VEGF domain is TFYDIETLKVIDEEWQRTQ and the second VEGF domain is CHPIETLVDIFQEYPDEIEYIFKPSCVPLMRCGGCCNDEG.

12. The chimeric synthetic protein according to claim 9, wherein the chimeric polypeptide sequence binds to a vascular endothelial growth factor receptor (VEGFR) selected from the group consisting of VEGFR-1, VEGFR-2, VEGFR-3, and combinations thereof.

13. The chimeric synthetic protein according to claim 12, wherein the chimeric polypeptide sequence binds to VEGFR-1, VEGFR-2, and VEGFR-3.

14. The chimeric synthetic protein according to claim 1, wherein the chimeric synthetic protein initially has an amino acid sequence of
MTPQNITDLCAEYHNTQIHTLNDKIFSYTESLAGKREMAIITFKNGATFQVEV
DSQKKAIERMKDTLRIAYLTEAKVEKLCVWNNKTPHAIAAISMANSSGGSGGG
PGSQHISGTFYDIETLKVIDEEWQRTQCHPIETLVDIFQEYPDEIEYIFKPSC
VPLMRCGGCCNDEG.

15. The chimeric synthetic protein according to claim 14, wherein the initial chimeric synthetic protein is processed to have an amino acid sequence of
TPQNITDLCAEYHNTQIHTLNDKIFSYTESLAGKREMAIITFKNGATFQVEVP
GSQHIDSQKKAIERMKDTLRIAYLTEAKVEKLCVWNNKTPHAIAAISMANSSG
GSGGGSGTFYDIETLKVIDEEWQRTQCHPIETLVDIFQEYPDEIEYIFKPSCV
PLMRCGGCCNDEG.

16. An immunogenic composition, comprising:
a chimeric polypeptide sequence;
at least one linker, and
a polypeptide sequence.

17. The immunogenic composition according to claim 16, wherein the polypeptide sequence includes an immunogenic polypeptide sequence.

18. The immunogenic composition according to claim 16, wherein the polypeptide sequence includes a cholera toxin B (CT-B) protein.

19. The immunogenic composition according to claim 16, wherein the at least one linker includes a first linker that separates the chimeric polypeptide sequence from the polypeptide sequence.

20. The immunogenic composition according to claim 16, wherein the first linker is selected from the group consisting of SSG, GSSG, SSGGG, SGG, GGSGG, GGGGS, SSGGGSGG, SSGGGGSGGG, TSGGGSG, TSGGGGSGG, SSGGSGGGSG, SSGGGSGGSSG, GGSGGTSGGGSG, SGGTSGGGGSGG, GGSGGTSGGGGSGG, SSGGGGSGGGSSG, SSGGGSGGSSGGG, and SSGGGGSGGGSSGGG.

21. The immunogenic composition according to claim 20, wherein the first linker is SSGGSGGGSG.

22. The immunogenic composition according to claim 16, wherein the chimeric polypeptide sequence includes a vascular endothelial growth factor (VEGF) sequence.

23. The immunogenic composition according to claim 16, wherein the chimeric polypeptide sequence includes a VEGF sequence selected from the group consisting of VEGF-A, VEGF-B, VEGF-C, VEGF-D, and combinations thereof.

24. The immunogenic composition according to claim 16, wherein the chimeric polypeptide sequence includes a first VEGF domain and a second VEGF domain.

25. The immunogenic composition according to claim 24, wherein the first VEGF domain includes VEGF-D, or a portion thereof, and the second VEGF domain includes VEGF-A, or a portion thereof.

26. The immunogenic composition according to claim 25, wherein the first VEGF domain is TFYDIETLKVIDEEWQRTQ and the second VEGF domain is CHPIETLVDIFQEYPDEIEYIFKPSCVPLMRCGGCCNDEG.

27. The immunogenic composition according to claim 26, wherein the chimeric polypeptide sequence binds to a vascular endothelial growth factor receptor (VEGFR) selected from the group consisting of VEGFR-1, VEGFR-2, VEGFR-3, and combinations thereof.

28. The immunogenic composition according to claim 27, wherein the chimeric polypeptide sequence binds to VEGFR-1, VEGFR-2, and VEGFR-3.

29. The immunogenic composition according to claim 16, wherein the synthetic protein chimeric synthetic protein initially has an amino acid sequence of
MTPQNITDLCAEYHNTQIHTLNDKIFSYTESLAGKREMAIITFKNGATFQVEV
PGSQHIDSQKKAIERMKDTLRIAYLTEAKVEKLCVWNNKTPHAIAAISMANSS
GGSGGGSGTFYDIETLKVIDEEWQRTQCHPIETLVDIFQEYPDEIEYIFKPSC
VPLMRCGGCCNDEG.

30. The immunogenic composition according to claim 16, wherein the initial chimeric synthetic protein is processed to have an amino acid sequence of
TPQNITDLCAEYHNTQIHTLNDKIFSYTESLAGKREMAIITFKNGATFQVEVP
GSQHIDSQKKAIERMKDTLRIAYLTEAKVEKLCVWNNKTPHAIAAISMANSSG
GSGGGSGTFYDIETLKVIDEEWQRTQCHPIETLVDIFQEYPDEIEYIFKPSCV
PLMRCGGCCNDEG.

31. The immunogenic composition according to claim 16, further comprising an adjuvant.

32. A method of treating a patient in need thereof, comprising:
administering to the patient the immunogenic composition of claim 16 in a same day or at alternate days or times during a vaccination period.

33. The method of claim 32, wherein the patient has a cancer.
